SEQUENCE LISTING (8 sequences)

SEQ ID NO: 1 - DNA, 15 bp, Artificial Sequence
  chimera decoy NF-kappaB/STAT6-15mer-B
gggatttcctgggaa

SEQ ID NO: 2 - DNA, 10 bp, Artificial Sequence
  binding region of NF-kappaB
gggatttcct

SEQ ID NO: 3 - DNA, 11 bp, Artificial Sequence
  binding region of STAT6
ttcccaggaaa

SEQ ID NO: 4 - DNA, 10 bp, Artificial Sequence
  consensus sequence of NF-kappaB
gggrhtyyhc

SEQ ID NO: 5 - DNA, 10 bp, Artificial Sequence
  consensus sequence of STAT6
  misc_feature (4)..(7): n is a, c, g, or t
ttcnnnngaa

SEQ ID NO: 6 - DNA, 15 bp, Artificial Sequence
  chimera decoy NF-kappaB/STAT6-15mer-A
gggacttcccatgaa

SEQ ID NO: 7 - DNA, 10 bp, Artificial Sequence
  partial sequence of NF-kappaB/STAT6-15mer-A
gggacttccc

SEQ ID NO: 8 - DNA, 11 bp, Artificial Sequence
  partial sequence of NF-kappaB/STAT6-15mer-A
ttcatgggaag

(19) United States
(12) Patent Application Publication
(10) Pub. No.: US 2023/0069350 A1
(43) Pub. Date: Mar. 2, 2023

(54) METHODS AND COMPOSITIONS RELATING TO PROLONGED INFLAMMATORY INHIBITION AND/OR PROLONGED TREATMENT OF SPINAL PAIN BY CHIMERA DECOY

(71) Applicants: The Regents of the University of California, Oakland, CA (US); AnGes, Inc., Osaka (JP)

(72) Inventors: Koichi Masuda, La Jolla, CA (US); Takahiro Nakazawa, Osaka (JP)

(21) Appl. No.: 17/793,377

(22) PCT Filed: Jan. 14, 2021
(86) PCT No.: PCT/US2021/013404

§ 371 Date: Jul. 15, 2022

Related U.S. Application Data
(60) Provisional application No. 62/961,405, filed on Jan. 15, 2020.

(51) Int. Cl. A61K 31/713; A61P 19/04

(52) U.S. Cl. CPC A61K 31/713; A61P 19/04

(57) ABSTRACT

Provided herein are methods and compositions for long term treatment of spinal pain. In particular, use of double-stranded oligonucleotide decoys capable of binding to the DNA binding sites of two transcription factors are provided for treatment of spinal pain for over seven days, where the decoys are administered without a drug delivery system.

### CROSS-REFERENCE TO RELATED APPLICATION

This application claims priority under 35 U.S.C. § 119(e) to U.S. Provisional Application No. 62/961,405, filed Jan. 15, 2020, the content of which is hereby incorporated by reference in its entirety.

### SEQUENCE LISTING

The instant application contains a Sequence Listing which has been submitted electronically in ASCII format and is hereby incorporated by reference in its entirety. Said ASCII copy, created on Jan. 11, 2021, is named 114198-7090_SL_ST25 and is 2,101 bytes in size.

### TECHNICAL FIELD

The disclosure relates generally to oligonucleotide decoys and more specifically to use of double-stranded oligonucleotide decoys capable of binding to the DNA binding sites of two transcription factors for prolonged treatment of inflammation and/or spinal pain.

### BACKGROUND

Low back pain (LBP) is a major cause of disability in the United States today. It is a common condition affecting many individuals at some point in their lives, has become one of the biggest problems for the public health system worldwide (Balague et al. Lancet. 2012; 379: 482-91). Over a three-year period, 15-20% of Americans require medical care for back pain. The cost to society of back pain is over fifty (50) billion dollars because of lost productivity and medical care expenses. LBP can be caused by many components of the lumbar spine, particularly the intervertebral discs (IVDs), the paravertebral muscles, and the zygapophyseal, or facet joint. Facet osteoarthritis (OA) is a major cause of low back pain. Recent studies have suggested that up to 30% of LBP is generated by the facet joint. Chondrocytes in cartilage actively regulate the homeostasis of the matrix metabolism by maintaining a balance between anabolism and catabolism: this balance is tightly controlled by a variety of substances, including cytokines, growth factors, enzymes and enzyme inhibitors in a paracrine and/or autocrine fashion. The involvement of pro-inflammatory cytokines has been shown in pathogenesis by both stimulating matrix-degrading enzymes and suppressing extracellular matrix production. These cytokines are also heavily involved in pain generation. Although it has recently been suggested that facet joint pathology causes LBP, historically, degeneration of the IVD has been associated with back pain and it is thought that IVD degeneration precedes facet joint osteoarthritis (OA) (Buckwalter J A et al. Spine. 1995; 20: 1307-14). However, the relationship between degenerative changes in the facet joint and the IVD are largely unknown.

Several different therapeutic approaches for facet OA can be considered by modulating the anabolic/catabolic balance at various points to reduce pain generation. Currently, most treatments of facet joint OA are limited to physical therapy, medial branch block, intra-articular local anesthesia, steroid injection or radiofrequency denervation. Based on the US Preventive Service Task Force (USPSTF) criteria, the evidence level for medial branch block is I˜II-1. The evidence levels for intra-articular facet joint blocks and radiofrequency denervation are II-1˜2 and II-2˜II-3, respectively. Surgery is also occasionally performed, although there is no clear evidence to support surgical intervention. Recently, the effects of hyaluronic acid for facet joint arthropathy were investigated, but the efficacy of this treatment remains controversial.

Inflammatory features are known to play a pivotal role in the progression of facet joint degeneration and pain generation. The inflammatory response also plays an important role in IVD degeneration (Frontana G et al. Adv Drug Deliv Rev. 2015; 84: 146-58). Pro-inflammatory cytokines, such as interleukin (IL)-1 and tumor necrosis factor-α (TNFα), have been considered possible candidates as key factors for proteoglycan (PG) loss in IVD degeneration. A one-year follow-up of a double-blinded, controlled clinical trial that used lumbar facet joint nerve blocks to treat chronic LBP generated in the facet joint suggests that non-surgical therapies can be used to manage these patients. Facet joint injection is a commonly used minimally invasive procedure that involves an injection of a corticosteroid; however, steroids are known to have side effects, such as suppression of matrix synthesis by chondrocytes and infection.

Accordingly, a need exists for a non-invasive therapy for treating LBP, such as facet joint pain.

### SUMMARY OF THE DISCLOSURE

The present disclosure is based on the finding that a single dose of a chimera decoy oligodeoxynucleotide is effective for more than seven days, for example, in one or more of the following: decreasing the gene expression of pro-inflammatory cytokines, suppressing an inflammatory response, treating intervertebral disc degeneration, regenerating chondrocyte extracellular matrix, promoting synthesis of proteoglycan, treating Facet osteoarthritis (OA), treating low back pain (LBP), and treating spinal pain and synovial joint pain.

Accordingly, in one aspect, the disclosure provides a method for the treatment of spinal pain. The method includes (for example, comprises, or consists essentially of, or further consists of) administering to a subject in need thereof a single dose of an effective amount of a double-stranded oligonucleotide decoy capable of binding to the DNA binding site of NF-κB and to the DNA binding site of signal transducer and activator of transcription 6 (STAT6), wherein the single dose is effective for over 1 week or longer, thereby treating spinal pain in the subject. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered by a method comprising, or consisting essentially of, or yet further consists of an intra-articular injection, optionally directly into a facet joint of the subject. In various embodiments, the decoy is administered via a method comprising, or consisting essentially of, or yet further consists of an intradiscal injection, or intervertebral disc injection, or epidural injection. In various embodiments, the single dose is effective for about 2 weeks or longer, such as 12 weeks.

In another aspect, the disclosure provides a method of treating intervertebral disc degeneration in a subject and synovial joint degeneration. The method includes (for example, comprises, or consists essentially of, or further consists of) administering to a subject in need thereof a single dose of an effective amount of a double-stranded oligonucleotide decoy capable of binding to the DNA binding site of NF-κB and to the DNA binding site of signal transducer and activator of transcription 6 (STAT6), wherein the single dose is effective for over 1 week or longer, thereby treating intervertebral disc degeneration or and synovial joint degeneration in the subject. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered via a method comprising, or consisting essentially of, or yet further consists of an intra-articular injection, optionally directly into a facet joint of the subject. In various embodiments, the decoy is administered via intradiscal injection, or intervertebral disc injection, or epidural injection. In various embodiments, the single dose is effective for about 2 weeks or longer, such as 12 weeks.

In another aspect, the disclosure provides a method for regenerating a chondrocyte extracellular matrix in a subject. The method includes (for example, comprises, or consists essentially of, or further consists of) administering to a subject in need thereof a single dose of an effective amount of a double-stranded oligonucleotide decoy capable of binding to the DNA binding site of NF-κB and to the DNA binding site of signal transducer and activator of transcription 6 (STAT6), wherein the single dose is effective for over 1 week or longer, thereby regenerating a chondrocyte extracellular matrix in the subject. In various embodiments, the chondrocyte extracellular matrix is an intervertebral disc cell extracellular matrix. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered via a method comprising, or consisting essentially of, or yet further consists of an intra-articular injection, optionally directly into a facet joint of the subject. In various embodiments, the decoy is administered via intradiscal injection, or intervertebral disc injection, or epidural injection. In various embodiments, the single dose is effective for about 2 weeks or longer, such as 12 weeks.

In another aspect, the disclosure provides a method for promoting the synthesis of proteoglycan in intervertebral disc cells of a subject. The method includes (for example, comprises, or consists essentially of, or further consists of) administering to a subject in need thereof a single dose of an effective amount of a double-stranded oligonucleotide decoy capable of binding to the DNA binding site of NF-κB and to the DNA binding site of signal transducer and activator of transcription 6 (STAT6), wherein the single dose is effective for over 1 week or longer, thereby promoting the synthesis of proteoglycan in intervertebral disc cells of the subject. In various embodiments, the intervertebral disc cells comprise, or consist essentially of, or further consist of nucleus pulposus cells and/or anulus fibrosus cells. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered via a method comprising, or consisting essentially of, or yet further consists of an intra-articular injection, optionally directly into a facet joint of the subject. In various embodiments, the decoy is administered via intradiscal injection, or intervertebral disc injection, or epidural injection. In various embodiments, the single dose is effective for about 2 weeks or longer, such as 12 weeks.

In yet another aspect, provided is a method for reducing and/or suppressing an inflammatory response. The method includes (for example, comprises, or consists essentially of, or further consists of) administering to a subject in need thereof a single dose of an effective amount of a double-stranded oligonucleotide decoy capable of binding to the DNA binding site of NF-κB and to the DNA binding site of signal transducer and activator of transcription 6 (STAT6), wherein the single dose is effective for over 1 week or longer, thereby reducing and/or suppressing an inflammatory response in the subject. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered via intra-articular injection, optionally directly into a facet joint of the subject. In various embodiments, the decoy is administered via intradiscal injection, or intervertebral disc injection, or epidural injection. In various embodiments, the single dose is effective for about 2 weeks or longer, such as 12 weeks. In various embodiments, the inflammatory response is in a target tissue or organ selected from one or both of: a lumbar spine (such as one or more of an intervertebral disc (such as either or both of annulus fibrosus and nucleus pulposus), a paravertebral muscle, or zygapophyseal), or a facet joint (such as synovial tissue or cartilaginous tissue).

Also provided is a method for the treatment of Facet osteoarthritis (OA). The method includes (for example, comprises, or consists essentially of, or further consists of) administering to a subject in need thereof a single dose of an effective amount of a double-stranded oligonucleotide decoy capable of binding to the DNA binding site of NF-κB and to the DNA binding site of signal transducer and activator of transcription 6 (STAT6), wherein the single dose is effective for over 1 week or longer, thereby treating the OA in the subject. In various embodiments, the decoy has a size of from 13 mer to 15 mer. In various embodiments, the decoy has a sequence represented by SEQ ID NO: 1 or 6. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered via intra-articular injection, optionally directly into a facet joint of the subject. In various embodiments, the single dose is effective for about 2 weeks or longer, such as 12 weeks.

In various embodiments of any aspect as disclosed herein, the single dose is effective for at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, or at least about 13 weeks, or at least about 14 weeks, or at least about 15 weeks, or at least about 4 months, or at least about 5 months, or at least about 6 months, or at least about 7 months, or at least about 8 months, or at least about 9 months, or at least about 1 year or longer. In various embodiments of any aspect as disclosed herein, the single dose is effective in one or more of the following: treating the intervertebral disc degeneration; regenerating a chondrocyte extracellular matrix; promoting the synthesis of proteoglycan in intervertebral disc cells; treating a spinal pain; treating a low back pain; reducing or suppressing an inflammatory response optionally in a target tissue or organ; decreasing the gene expression of a pro-inflammatory cytokine (optionally selected from IL-1, IL-1β, IL-6 or TNFα); treating Facet osteoarthritis (OA); reducing expression or production one or more of: a pain-related molecules (optionally selected from vascular endothelial growth factor (VEGF), prostaglandin-endoperoxide synthase 2 (PTGS2), and nerve growth factor (NGF)), a catabolic molecule (optionally ADAM Metallopeptidase With Thrombospondin Type 1 Motif 4 (ADAMTS4)), Matrix Metallopeptidase 3 (MMP3), prostaglandin E2 (PGE2), or nitric oxide (NO).

### BRIEF DESCRIPTION OF THE DRAWINGS

FIG. 1 is a pictorial diagram showing a service of microscopic images of an exemplary pellet.

FIG. 2 is a graphical diagram showing luciferase activity.

FIGS. 3A-3D provide results relating to the NF-κB reporter cell line. FIG. 3A shows result of fluorescent C-Decoy transfection in monolayer. FIG. 3B provides luminescence normalized by Renilla in Renilla-Firefly Luciferase Dual Assay. FIG. 3C shows the LPS effect (LPS+/LPS− of each group) as the ratio to control (PBS). FIG. 3D provides microscopic images of pellet.

FIGS. 4A-4C show effects of C-decoy in rabbit articular cartilage. FIG. 4A shows the rate of proteoglycan turnover. Decoy ODNs restored PG turnover stimulated by IL-1β. The line starting at 100% remaining ³⁵PG in the tissue represents the control (Ctrl), while the line starting right below the control line represents the Chimera (10 μM)+IL1 treatment group. The line immediately below the control line and the Chimera (10 μM)+IL1 line represents the treatment group by NF-κB decoy (Decoy) (10 μM)+IL. The bottom line represents the IL1 only treatment group, while the line immediately above the IL1-only line represents the Chimera (1 μM)+IL1 treatment group. FIG. 4B provides NO productions. 10 μM Chimer decoy reduced NO production stimulated by IL-10 at day 2 and day 4. For each day, four columns are presented (from left to right, cont, IL1, Chimera (1)+IL1, and Chimera (10)+IL1). FIG. 4C provides MMP3 production. Media at day 2 were measured. Four groups are plotted (from left to right, control (0 0), IL1 (0 5), Chimera (1 μM)+IL1, and Chimera (10 μM)+IL1). Chimer decoy significantly reduces MMP3 production stimulated by IL-1β.

FIGS. 5A-5B show effects of NF-κB decoy on proteoglycan (PG) plotted provides PG synthesis (FIG. 5A) and turnover (FIG. 5B) by facet joint cartilage explants cultures (average±SE, Kakutani K et al. Ortho Res Soc Trans:1781, 2009).

FIGS. 6A-6B show normalized gene expression in synovial explant (FIG. 6A) and protein levels in the culture media (FIG. 6B) treated with PBS (left column of each group) or Chimera decoy (right column of each group). Data are expressed as mean and standard error (SE). *P<0.05; **P<0.01.

FIG. 7 shows reparative score at week 16. For each group, four columns are presented (from left to right, PBS only, 100 μg NF-κB decoy, 10 μg Chimera decoy, and 100 μg Chimera decoy).

### DETAILED DESCRIPTION OF THE DISCLOSURE

The present disclosure is based on the finding that a single dose of a chimera decoy oligodeoxynucleotide is effective, for example, in one or more of the following: suppressing an inflammatory response, treating intervertebral disc degeneration, regenerating chondrocyte extracellular matrix, promoting synthesis of proteoglycan, treating spinal pain, treating Facet osteoarthritis (OA), treating or preventing synovial joint degeneration or pain, and/or treating low back pain (LBP) for more than seven days. Without wishing to be bound by the theory, the instant disclosure inhibits cytokines by the injection of cytokine or signaling pathway inhibitors, thus slowing matrix catabolism and stimulating the matrix anabolic pathway. Miyazaki S et al. (Trans Orthop Res Soc:0387, 2018) indicated that an in vitro 24 hour pre-treatment of synovial tissues with Chimera Decoy (CDecoy) inhibited the inflammatory responses during the subsequent 48 hours. However, the long-term effects of a single transfection was not disclosed. As shown in the Examples, a successful transfection of fluorescent decoy was confirmed, and a single transfection of chimera decoy was effective over one week without any carrier or virus. In some embodiments, no drug delivery system is not required for the long term anti-inflammatory effect as disclosed herein, thus reducing and/or eliminating the cost and development burden.

Accordingly, in one aspect, provided are methods pertaining to the use of a chimera decoy for prolonged treatment of spinal pain or and synovial joint degeneration or pain. In some embodiments, a single injection of Chimera Decoy (intradiscal or intra-articular) is effective over a long period of time, such as one week or two weeks. In further embodiments, the injection is effective without a drug delivery system (such as a carrier or virus) over one week, which reduces or eliminates the cost and development burden. In some embodiments, drug delivery system is not required for the long term anti-inflammatory effect.

### Definitions

As it would be understood, the section or subsection headings as used herein is for organizational purposes only and are not to be construed as limiting and/or separating the subject matter described.

Before the present compositions and methods are described, it is to be understood that this disclosure is not limited to particular compositions, methods, and experimental conditions described, as such compositions, methods, and conditions may vary. It is also to be understood that the terminology used herein is for purposes of describing particular embodiments only, and is not intended to be limiting, since the scope of the present disclosure will be limited only in the appended claims.

As used in this specification and the appended claims, the singular forms “a”, “an”, and “the” include plural references unless the context clearly dictates otherwise. Thus, for example, references to “the method” includes one or more methods, and/or steps of the type described herein which will become apparent to those persons skilled in the art upon reading this disclosure and so forth.

The term “comprising,” which is used interchangeably with “including,” “containing,” or “characterized by,” is inclusive or open-ended language and does not exclude additional, unrecited elements or method steps. The phrase “consisting of” excludes any element, step, or ingredient not specified in the claim. The phrase “consisting essentially of” limits the scope of a claim to the specified materials or steps and those that do not materially affect the basic and novel characteristics of the claimed invention. The present disclosure contemplates embodiments of the invention compositions and methods corresponding to the scope of each of these phrases. Thus, a composition or method comprising recited elements or steps contemplates particular embodiments in which the composition or method consists essentially of or consists of those elements or steps.

“Optional” or “optionally” means that the subsequently described circumstance may or may not occur, so that the description includes instances where the circumstance occurs and instances where it does not.

As used herein, “and/or” refers to and encompasses any and all possible combinations of one or more of the associated listed items, as well as the lack of combinations when interpreted in the alternative (“or”).

All numerical designations, e.g., pH, temperature, time, concentration, and molecular weight, including ranges, are approximations which are varied (+) or (−) by increments of 1.0 or 0.1, as appropriate, or alternatively by a variation of +/−15%, or alternatively 10%, or alternatively 5%, or alternatively 2%. It is to be understood, although not always explicitly stated, that all numerical designations are preceded by the term “about”. It also is to be understood, although not always explicitly stated, that the reagents described herein are merely exemplary and that equivalents of such are known in the art.

It is to be inferred without explicit recitation and unless otherwise intended, that when the present technology relates to a polypeptide, protein, polynucleotide or antibody, an equivalent or a biologically equivalent of such is intended within the scope of the present technology.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present technology, the preferred methods, devices and materials are now described.

The practice of the present technology will employ, unless otherwise indicated, conventional techniques of tissue culture, immunology, molecular biology, microbiology, cell biology, and recombinant DNA, which are within the skill of the art. See, e.g., Green and Sambrook eds. (2012) Molecular Cloning: A Laboratory Manual, 4^{th} edition; the series Ausubel et al. eds. (2015) Current Protocols in Molecular Biology; the series Methods in Enzymology (Academic Press, Inc., N.Y.); MacPherson et al. (2015) PCR 1: A Practical Approach (IRL Press at Oxford University Press); MacPherson et al. (1995) PCR 2: A Practical Approach; McPherson et al. (2006) PCR: The Basics (Garland Science); Harlow and Lane eds. (1999) Antibodies, A Laboratory Manual; Greenfield ed. (2014) Antibodies, A Laboratory Manual; Freshney (2010) Culture of Animal Cells: A Manual of Basic Technique, 6^{th} edition; Gait ed. (1984) Oligonucleotide Synthesis; U.S. Pat. No. 4,683,195; Hames and Higgins eds. (1984) Nucleic Acid Hybridization; Anderson (1999) Nucleic Acid Hybridization; Herdewijn ed. (2005) Oligonucleotide Synthesis: Methods and Applications; Hames and Higgins eds. (1984) Transcription and Translation; Buzdin and Lukyanov ed. (2007) Nucleic Acids Hybridization: Modern Applications; Immobilized Cells and Enzymes (IRL Press (1986)); Grandi ed. (2007) In Vitro Transcription and Translation Protocols, 2^{nd} edition; Guisan ed. (2006) Immobilization of Enzymes and Cells; Perbal (1988) A Practical Guide to Molecular Cloning, 2^{nd} edition; Miller and Calos eds, (1987) Gene Transfer Vectors for Mammalian Cells (Cold Spring Harbor Laboratory); Makrides ed. (2003) Gene Transfer and Expression in Mammalian Cells; Mayer and Walker eds. (1987) Immunochemical Methods in Cell and Molecular Biology (Academic Press, London); Lundblad and Macdonald eds. (2010) Handbook of Biochemistry and Molecular Biology, 4^{th} edition; and Herzenberg et al. eds (1996) Weir's Handbook of Experimental Immunology, 5^{th} edition; and the more recent editions each thereof available at the time of filing.

All publications, patents, and patent applications mentioned in this specification are herein incorporated by reference in their entireties to the same extent as if each individual publication, patent, or patent application was specifically and individually indicated to be incorporated by reference. Nothing herein is to be construed as an admission that the present technology is not entitled to antedate such disclosure by virtue of prior invention.

As used herein, the term “decoy” refers to a structure that resembles one to which a certain substance should originally bind or act. As provided herein, a decoy used for a transcription factor may be a double-stranded oligonucleotide having the same DNA sequence as the binding region of the transcription factor on a genome gene. In the presence of such an oligonucleotide decoy, some of the transcription factor molecules bind to the decoy oligonucleotide, instead of binding to the binding region on the genomic gene to which it should have bound. This results in a decrease in the number of transcription factor molecules that bind to the binding region on the genomic gene to which it should have bound, leading to a decrease in the activity of the transcription factor. A chimeric decoy contains DNA sequences for binding more than one transcription factor.

As used herein, the term “allodynia” refers to central pain sensitization (increased response of neurons) following normally non-painful, often repetitive, stimulation. Allodynia can lead to the triggering of a pain response from stimuli which do not normally provoke pain.

As used herein, “discogenic pain” refers to pain originating from a damaged vertebral disc, particularly due to degenerative disc disease.

Intervertebral discs (IVDs) of the spine consist of an outer annulus fibrosus (AF), which is rich in collagens that account for their tensile strength, and an inner nucleus pulposus (NP), which contains large proteoglycans (PGs) that retain water for resisting compression loading. Biologically, disc cells in both the AF and NP maintain a balance between anabolism and catabolism, or steady state metabolism, of their extracellular matrices (ECMs), and are modulated by a variety of substances including cytokines, enzymes, their inhibitors and growth factors such as insulin like growth factor (IGF), transforming growth factor β (TGF-β), and bone morphogenetic proteins (BMPs). Various enzymes, such as matrix metalloproteinases (MMPs), and cytokines, mediate the catabolic process, or breakdown of the matrix. The degeneration of an IVD is thought to result from an imbalance between the anabolic and catabolic processes, or the loss of steady state metabolism, that are maintained in the normal disc.

As used herein, “facet joints” refer to the joints in a spine that make the back flexible and enable a subject to bend and twist. Nerves exit the spinal cord through these joints on their way to other parts of the body. Healthy facet joints have cartilage, which allows the vertebrae to move smoothly against each other without grinding.

The term “subject”, “patient” or “host organism,” as used herein interchangeably, refers to any individual or patient to which the subject methods are performed. Generally the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., sport animals, pets, and primates (including monkeys, chimpanzees, orangutans and gorillas) are included within the definition of subject. Besides being useful for human treatment, the present disclosure is also useful for veterinary treatment of companion mammals, exotic animals and domesticated animals, including mammals, rodents. In one embodiment of the present disclosure, the human is a fetus, an infant, a pre-pubescent subject, an adolescent, a pediatric patient, or an adult. In one aspect, the subject is pre-symptomatic mammal or human. In another embodiment, the subject has minimal clinical symptoms of the disease. The subject can be a male or a female, adult, an infant or a pediatric subject. In an additional aspect, the subject is an adult. In some instances, the adult is an adult human, e.g., an adult human greater than 18 years of age.

Chondrocyte, as used herein, refers to cells in cartilage. A chondrocyte free of a disease produces and maintains the cartilaginous matrix, which consists mainly of collagen and proteoglycans (PGs). Mesenchymal stem cells are a naturally occurring progenitor of chondrocytes. Other stem cells may derive chondrocytes under suitable conditions, a non-limiting example of which is provided in the Examples.

As used herein, the term “animal” refers to living multi-cellular vertebrate organisms, a category that includes, for example, mammals and birds. The term “mammal” includes both human and non-human mammals.

The term “therapeutically effective amount” or “effective amount” means the amount of a compound or pharmaceutical composition that elicits the biological or medical response of a tissue, system, animal or human that is being sought by the researcher, veterinarian, medical doctor or other clinician. Thus, the term “therapeutically effective amount” is used herein to denote any amount of a formulation that causes a substantial improvement in a disease condition when applied to the affected areas repeatedly over a period of time. The amount varies with the condition being treated (such as the age, weight, etc., of the subject to be treated), the stage of advancement of the condition, and the type and concentration of formulation applied. Appropriate amounts in any given instance will be readily apparent to those skilled in the art or capable of determination by routine experimentation.

As used herein, the terms “reduce” and “inhibit” are used together because it is recognized that, in some cases, a decrease can be reduced below the level of detection of a particular assay, for example by about 5%, about 10%, about 15%, about 20%, about 25%, about 30%, about 40%, about 50%, about 60%, about 70%, about 80%, about 85%, about 90%, about 95%, about 99%, or about 100% of the reference level. As such, it may not always be clear whether the expression level or activity is “reduced” below a level of detection of an assay, or is completely “inhibited.” Nevertheless, it will be clearly determinable, following a treatment according to the present methods.

As used herein, “treatment” or “treating” means to administer a composition to a subject or a system with an undesired condition obtaining a desired pharmacologic and/or physiologic effect. Examples of “treatment” include but are not limited to: preventing a disorder from occurring in a subject that may be predisposed to a disorder, but has not yet been diagnosed as having it; inhibiting a disorder, i.e., arresting its development; and/or relieving or ameliorating the symptoms of disorder. In one aspect, treatment is the arrestment of the development of symptoms of the disease or disorder, such as a cancer. In some embodiments, it refers to (1) preventing the symptoms or disease from occurring in a subject that is predisposed or does not yet display symptoms of the disease; (2) inhibiting the disease or arresting its development; or (3) ameliorating or causing regression of the disease or the symptoms of the disease. As understood in the art, “treatment” is an approach for obtaining beneficial or desired results, including clinical results. For the purposes of the present technology, beneficial or desired results can include one or more, but are not limited to, alleviation or amelioration of one or more symptoms, diminishment of extent of a condition (including a disease), stabilized (i.e., not worsening) state of a condition (including disease), delay or slowing of condition (including disease), progression, amelioration or palliation of the condition (including disease), states and remission (whether partial or total), whether detectable or undetectable. Treatments containing the disclosed compositions and methods can be first line, second line, third line, fourth line, fifth line therapy and are intended to be used as a sole therapy or in combination with other appropriate therapies. In one aspect, the term “treatment” or “treating” excludes prevention or prophylaxis.

“Prevention” or “preventing” means to administer a composition to a subject or a system at risk for the condition. The condition can include a predisposition to a disease or disorder. The effect of the administration of the composition to the subject (either treating and/or preventing) can be, but is not limited to, the cessation of one or more symptoms of the condition, a reduction or prevention of one or more symptoms of the condition, a reduction in the severity of the condition, the complete ablation of the condition, a stabilization or delay of the development or progression of a particular event or characteristic, or minimization of the chances that a particular event or characteristic will occur.

The phrase “first line” or “second line” or “third line” refers to the order of treatment received by a patient. First line therapy regimens are treatments given first, whereas second or third line therapy are given after the first line therapy or after the second line therapy, respectively.

The condition can include a condition, disease or disorder. In one embodiment, the term “disease” or “disorder” as used herein refers to one or more of spinal pain, low back pain (LBP), intervertebral disc (IVD) degeneration, facet osteoarthritis (OA), chondrocyte extracellular matrix degeneration, reduced and/or inhibited synthesis of proteoglycan for example in intervertebral disc cells, inflammation response, or any other cause of spinal pain and/or low back pain; a status of being diagnosed with such disease; a status of being suspect of having such disease; or a status of at high risk of having such disease.

An inflammatory response, as used herein interchangeably with inflammation, refers to an immune response that occurs when tissues are injured by bacteria, trauma, toxins, heat, or any other cause. The damaged tissue releases compounds including histamine, bradykinin, and serotonin. Inflammation refers to both acute responses (i.e., responses in which the inflammatory processes are active) and chronic responses (i.e., responses marked by slow progression and formation of new connective tissue). Acute and chronic inflammation can be distinguished by the cell types involved. Acute inflammation often involves polymorphonuclear neutrophils; whereas chronic inflammation is normally characterized by a lymphohistiocytic and/or granulomatous response. Inflammation includes reactions of both the specific and non-specific defense systems. A specific defense system reaction is a specific immune system reaction response to an antigen (possibly including an autoantigen). A non-specific defense system reaction is an inflammatory response mediated by leukocytes incapable of immunological memory and/or cytokines. Such cells include granulocytes, macrophages, neutrophils and eosinophils. Examples of specific types of inflammation are diffuse inflammation, focal inflammation, croupous inflammation, interstitial inflammation, obliterative inflammation, parenchymatous inflammation, reactive inflammation, specific inflammation, toxic inflammation and traumatic inflammation.

In some embodiments, the inflammatory response causes, at least in part, one or more diseases as described herein, such as spinal pain, low back pain (LBP), intervertebral disc (IVD) degeneration, facet osteoarthritis (OA), chondrocyte extracellular matrix degeneration, and/or reduced and/or inhibited synthesis of proteoglycan for example in intervertebral disc cells. Additionally or alternatively, the inflammatory response is an inflammation of a target tissue and/or organ, such as a lumbar spine, such as intervertebral discs (IVDs), paravertebral muscles, and zygapophyseal, or a facet joint. In some embodiments, the inflammatory response is only within one or more of the target tissues and/or organs, but is not a systematic and/or diffuse inflammation.

In some embodiments, an inflammatory response comprises, or consists essentially of, or further consists of gene expression of one or more pro-inflammatory cytokines. In further embodiments, reducing and/or suppressing an inflammatory response comprises, or consists essentially of, or further consists of reducing and/or suppressing gene expression of one or more pro-inflammatory cytokines. As used herein, pro-inflammatory cytokines, such as interleukin (IL)-1, IL-1β, IL-6 and tumor necrosis factor-α (TNFα), are produced by an immune cell (such as activated macrophage), a target tissue/organ, and/or a chondrocyte, and are involved in the up-regulation of inflammation.

As used herein, a target tissue/organ refers to a tissue/organ showing symptom of a disease as disclosed herein and/or causing a disease as disclosed herein. In some embodiments, an inflammatory response in a target tissue/organ causes, at least in part, a disease as disclosed herein. Additionally or alternatively, a target tissue/organ is the target of a treatment method as disclosed herein. In some embodiments, a target tissue/organ is selected from one or more of a lumbar spine, such as intervertebral discs (IVDs), paravertebral muscles, and zygapophyseal, or a facet joint. In further embodiments, a target tissue/organ comprises, or consists essentially of, or further consists of annulus fibrosus, which is the tough circular exterior of the intervertebral disc that surrounds the soft inner core, the nucleus pulposus. Additionally or alternatively, a target tissue/organ comprises, or consists essentially of, or further consists of the nucleus pulposus of the intervertebral disc. In yet further embodiments, a target tissue/organ comprises, or consists essentially of, or further consists of synovial tissue and/or cartilaginous tissue, such as of the facet joint. In some embodiments, a target tissue/organ comprises, or consists essentially of, or further consists of chondrocytes.

The terms “polypeptide,” “peptide,” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer. The residues may be linked by peptide bonds. In another aspect, the subunit may be linked by other bonds, e.g., ester, ether, etc. A protein or peptide must contain at least two amino acids and no limitation is placed on the maximum number of amino acids which may comprise a protein's or peptide's sequence.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, α-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid. As used herein the term “amino acid” refers to both the D and L optical isomers.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

As used herein, the term “gene” means the deoxyribonucleotide sequences comprising the coding region of a structural gene. A “gene” may also include non-translated sequences located adjacent to the coding region on both the 5′ and 3′ ends such that the gene corresponds to the length of the full-length mRNA. The sequences which are located 5′ of the coding region and which are present on the mRNA are referred to as 5′ non-translated sequences. The sequences which are located 3′ or downstream of the coding region and which are present on the mRNA are referred to as 3′ non-translated sequences. The term “gene” encompasses both cDNA and genomic forms of a gene. A genomic form or clone of a gene contains the coding region interrupted with non-coding sequences termed “introns” or “intervening regions” or “intervening sequences.” Introns are segments of a gene which are transcribed into heterogenous nuclear RNA (hnRNA); introns may contain regulatory elements such as enhancers. Introns are removed or “spliced out” from the nuclear or primary transcript; introns therefore are absent in the messenger RNA (mRNA) transcript. The mRNA functions during translation to specify the sequence or order of amino acids in a nascent polypeptide.

A “sequence” of a nucleic acid refers to the order and identity of nucleotides in the nucleic acid. A sequence is typically read in the 5′ to 3′ direction. The terms “identical” or percent “identity” in the context of two or more nucleic acid or polypeptide sequences, refer to two or more sequences or subsequences that are the same or have a specified percentage of amino acid residues or nucleotides that are the same, when compared and aligned for maximum correspondence, e.g., as measured using one of the sequence comparison algorithms available to persons of skill or by visual inspection. Exemplary algorithms that are suitable for determining percent sequence identity and sequence similarity are the BLAST programs, which are described in, e.g., Altschul et al. (1990) “Basic local alignment search tool” J. Mol. Biol. 215:403-410, Gish et al. (1993) “Identification of protein coding regions by database similarity search” Nature Genet. 3:266-272, Madden et al. (1996) “Applications of network BLAST server” Meth. Enzymol. 266:131-141, Altschul et al. (1997) “Gapped BLAST and PSI-BLAST: a new generation of protein database search programs” Nucleic Acids Res. 25:3389-3402, and Zhang et al. (1997) “PowerBLAST: A new network BLAST application for interactive or automated sequence analysis and annotation” Genome Res. 7:649-656, which are each incorporated by reference. Many other optimal alignment algorithms are also known in the art and are optionally utilized to determine percent sequence identity.

As used herein, the terms “functionally linked” and “operably linked” are used interchangeably and refer to a functional relationship between two or more DNA segments, in particular gene sequences to be expressed and those sequences controlling their expression. For example, a promoter/enhancer sequence, including any combination of cis-acting transcriptional control elements is operably linked to a coding sequence if it stimulates or modulates the transcription of the coding sequence in an appropriate host cell or other expression system. Promoter regulatory sequences that are operably linked to the transcribed gene sequence are physically contiguous to the transcribed sequence.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the disclosure.

As used herein, the term “vector” refers to a nucleic acid construct deigned for transfer between different hosts, including but not limited to a plasmid, a virus, a cosmid, a phage, a BAC, a YAC, etc. In some embodiments, the vector is a non-viral vector, such as plasmid vectors which may be prepared from commercially available vectors. In other embodiments, the vector is a viral vector, such as those produced from baculoviruses, retroviruses, adenoviruses, AAVs, etc. according to techniques known in the art. In one embodiment, the viral vector is a lentiviral vector.

A “viral vector” is defined as a recombinantly produced virus or viral particle that comprises a polynucleotide to be delivered into a host cell, either in vivo, ex vivo or in vitro. Examples of viral vectors include retroviral vectors, lentiviral vectors, adenovirus vectors, adeno-associated virus vectors, alphavirus vectors and the like. Alphavirus vectors, such as Semliki Forest virus-based vectors and Sindbis virus-based vectors, have also been developed for use in gene therapy and immunotherapy. See, Schlesinger and Dubensky (1999) Curr. Opin. Biotechnol. 5:434-439 and Ying, et al. (1999) Nat. Med. 5(7):823-827.

In aspects where gene transfer is mediated by a lentiviral vector, a vector construct refers to the polynucleotide comprising the lentiviral genome or part thereof, and a therapeutic gene. As used herein, “lentiviral mediated gene transfer” or “lentiviral transduction” carries the same meaning and refers to the process by which a gene or nucleic acid sequences are stably transferred into the host cell by virtue of the virus entering the cell and integrating its genome into the host cell genome. The virus can enter the host cell via its normal mechanism of infection or be modified such that it binds to a different host cell surface receptor or ligand to enter the cell. Retroviruses carry their genetic information in the form of RNA; however, once the virus infects a cell, the RNA is reverse-transcribed into the DNA form which integrates into the genomic DNA of the infected cell. The integrated DNA form is called a provirus. As used herein, lentiviral vector refers to a viral particle capable of introducing exogenous nucleic acid into a cell through a viral or viral-like entry mechanism. A “lentiviral vector” is a type of retroviral vector well-known in the art that has certain advantages in transducing nondividing cells as compared to other retroviral vectors. See, Trono D. (2002) Lentiviral vectors, New York: Spring-Verlag Berlin Heidelberg.

Lentiviral vectors of this disclosure are based on or derived from oncoretroviruses (the sub-group of retroviruses containing MLV), and lentiviruses (the sub-group of retroviruses containing HIV). Examples include ASLV, SNV and RSV all of which have been split into packaging and vector components for lentiviral vector particle production systems. The lentiviral vector particle according to the disclosure may be based on a genetically or otherwise (e.g. by specific choice of packaging cell system) altered version of a particular retrovirus.

That the vector particle according to the disclosure is “based on” a particular retrovirus means that the vector is derived from that particular retrovirus. The genome of the vector particle comprises components from that retrovirus as a backbone. The vector particle contains essential vector components compatible with the RNA genome, including reverse transcription and integration systems. Usually these will include gag and pol proteins derived from the particular retrovirus. Thus, the majority of the structural components of the vector particle will normally be derived from that retrovirus, although they may have been altered genetically or otherwise so as to provide desired useful properties. However, certain structural components and in particular the env proteins, may originate from a different virus. The vector host range and cell types infected or transduced can be altered by using different env genes in the vector particle production system to give the vector particle a different specificity.

The terms “polynucleotide” and “oligonucleotide” are used interchangeably and refer to a polymeric form of nucleotides of any length, either deoxyribonucleotides or ribonucleotides or analogs thereof. Polynucleotides can have any three-dimensional structure and may perform any function, known or unknown. The following are non-limiting examples of polynucleotides: a gene or gene fragment (for example, a probe, primer, EST or SAGE tag), exons, introns, messenger RNA (mRNA), transfer RNA, ribosomal RNA, RNAi, ribozymes, cDNA, recombinant polynucleotides, branched polynucleotides, plasmids, vectors, isolated DNA of any sequence, isolated RNA of any sequence, nucleic acid probes and primers. A polynucleotide can comprise modified nucleotides, such as methylated nucleotides and nucleotide analogs. If present, modifications to the nucleotide structure can be imparted before or after assembly of the polynucleotide. The sequence of nucleotides can be interrupted by non-nucleotide components. A polynucleotide can be further modified after polymerization, such as by conjugation with a labeling component. The term also refers to both double- and single-stranded molecules. Unless otherwise specified or required, any aspect of this technology that is a polynucleotide encompasses both the double-stranded form and each of two complementary single-stranded forms known or predicted to make up the double-stranded form.

“Detectable label”, “label”, “detectable marker” or “marker” are used interchangeably, including, but not limited to radioisotopes, fluorochromes, chemiluminescent compounds, dyes, and proteins, including enzymes. Detectable labels can also be attached to a polynucleotide, polypeptide, antibody or composition described herein. In some embodiments relating to gene expression of a detectable maker, such detectable maker may be a polynucleotide transcribed from a gene, and/or a peptide/protein (such as an enzyme and/or a fluorescent protein) translated from a gene.

As used herein, the term “detectable marker” refers to at least one marker capable of directly or indirectly, producing a detectable signal. A non-exhaustive list of this marker includes enzymes which produce a detectable signal, for example by colorimetry, fluorescence, luminescence, such as horseradish peroxidase, alkaline phosphatase, β-galactosidase, glucose-6-phosphate dehydrogenase, chromophores such as fluorescent, luminescent dyes, groups with electron density detected by electron microscopy or by their electrical property such as conductivity, amperometry, voltammetry, impedance, detectable groups, for example whose molecules are of sufficient size to induce detectable modifications in their physical and/or chemical properties, such detection may be accomplished by optical methods such as diffraction, surface plasmon resonance, surface variation, the contact angle change or physical methods such as atomic force spectroscopy, tunnel effect, or radioactive molecules such as ³²P, ³⁵S or ¹²⁵I. The term also includes sequences conjugated to the polynucleotide that will provide a signal upon expression of the inserted sequences, such as green fluorescent protein (GFP) and the like. The label may be detectable by itself (e.g., radioisotope labels or fluorescent labels) or, in the case of an enzymatic label, may catalyze chemical alteration of a substrate compound or composition which is detectable. The labels can be suitable for small scale detection or more suitable for high-throughput screening. As such, suitable labels include, but are not limited to magnetically active isotopes, non-radioactive isotopes, radioisotopes, fluorochromes, chemiluminescent compounds, dyes, and proteins, including enzymes. The label may be simply detected or it may be quantified. A response that is simply detected generally comprises a response whose existence merely is confirmed, whereas a response that is quantified generally comprises a response having a quantifiable (e.g., numerically reportable) value such as an intensity, polarization, and/or other property. In luminescence or fluorescence assays, the detectable response may be generated directly using a luminophore or fluorophore associated with an assay component actually involved in binding, or indirectly using a luminophore or fluorophore associated with another (e.g., reporter or indicator) component. Examples of luminescent labels that produce signals include, but are not limited to bioluminescence and chemiluminescence. Detectable luminescence response generally comprises a change in, or an occurrence of a luminescence signal. Suitable methods and luminophores for luminescently labeling assay components are known in the art and described for example in Haugland, Richard P. (1996) Handbook of Fluorescent Probes and Research Chemicals (6th ed). Examples of luminescent probes include, but are not limited to, aequorin and luciferases.

Examples of suitable fluorescent labels include, but are not limited to, fluorescein, rhodamine, tetramethylrhodamine, eosin, erythrosin, coumarin, methyl-coumarins, pyrene, Malacite green, stilbene, Lucifer Yellow, Cascade Blue™, and Texas Red. Other suitable optical dyes are described in the Haugland, Richard P. (1996) Handbook of Fluorescent Probes and Research Chemicals (6th ed.).

A “composition” typically intends a combination of the active agent, e.g., compound or composition, and a naturally-occurring or non-naturally-occurring carrier, inert (for example, a detectable agent or label) or active, such as an adjuvant, diluent, binder, stabilizer, buffers, salts, lipophilic solvents, preservative, adjuvant or the like and include pharmaceutically acceptable carriers. Carriers also include pharmaceutical excipients and additives proteins, peptides, amino acids, lipids, and carbohydrates (e.g., sugars, including monosaccharides, di-, tri-, tetra-oligosaccharides, and oligosaccharides; derivatized sugars such as alditols, aldonic acids, esterified sugars and the like; and polysaccharides or sugar polymers), which can be present singly or in combination, comprising alone or in combination 1-99.99% by weight or volume. Exemplary protein excipients include serum albumin such as human serum albumin (HSA), recombinant human albumin (rHA), gelatin, casein, and the like. Carbohydrate excipients are also intended within the scope of this technology, examples of which include but are not limited to monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and myoinositol.

As used herein “pharmaceutically acceptable carrier” encompasses any of the standard pharmaceutical carriers, such as a phosphate buffered saline solution, water and emulsions such as an oil/water or water/oil emulsion, and various types of wetting agents. The compositions also can include stabilizers and preservatives. For examples of carriers, stabilizers and adjuvants, see Martin (1975) Remington's Pharm. Sci., 15th Ed. (Mack Publ. Co., Easton). In some embodiments, the term pharmaceutically acceptable carrier (or medium), which may be used interchangeably with the term biologically compatible carrier or medium, refers to reagents, cells, compounds, materials, compositions, and/or dosage forms that are not only compatible with the cells and other agents to be administered therapeutically, but also are, within the scope of sound medical judgment, suitable for use in contact with the tissues of human beings and animals without excessive toxicity, irritation, allergic response, or other complication commensurate with a reasonable benefit/risk ratio. Pharmaceutically acceptable carriers suitable for use in the present disclosure include liquids, semi-solid (e.g., gels) and solid materials (e.g., cell scaffolds and matrices, tubes sheets and other such materials as known in the art and described in greater detail herein). These semi-solid and solid materials may be designed to resist degradation within the body (non-biodegradable) or they may be designed to degrade within the body (biodegradable, bioerodable). A biodegradable material may further be bioresorbable or bioabsorbable, i.e., it may be dissolved and absorbed into bodily fluids (water-soluble implants are one example), or degraded and ultimately eliminated from the body, either by conversion into other materials or breakdown and elimination through natural pathways.

A “pharmaceutical composition” is intended to include the combination of an active compound, such as a decoy as disclosed herein, with a carrier, for example a pharmaceutically acceptable carrier, inert or active such as a solid support, making the composition suitable for diagnostic or therapeutic use in vitro, in vivo or ex vivo.

“Administration” or “delivery” of a compound, such as a decoy as disclosed herein, and compositions containing same can be effected in one dose, continuously or intermittently throughout the course of treatment. Methods of determining the most effective means and dosage of administration are known to those of skill in the art and will vary with the composition used for therapy, the purpose of the therapy, the target cell (such as a chondrocyte) being treated, and the subject being treated. Single or multiple administrations can be carried out with the dose level and pattern being selected by the treating physician or in the case of animals, by the treating veterinarian. Suitable dosage formulations and methods of administering the agents are known in the art. Route of administration can also be determined and method of determining the most effective route of administration are known to those of skill in the art and will vary with the composition used for treatment, the purpose of the treatment, the health condition or disease stage of the subject being treated, and target cell or tissue. Non-limiting examples of route of administration include oral administration, intraperitoneal, infusion, nasal administration, inhalation, injection, and topical application. In some embodiments, the route of administration is selected from one or more of intradiscal injection, or intra-articular injection, or intervertebral disc injection, or epidural injection.

### MODES FOR CARRYING OUT THE DISCLOSURE

### Decoys

Various double-stranded oligonucleotide decoys showing binding affinity for a transcription factor are known for treating or preventing diseases caused by a transcription factor by administering a decoy for the transcription factor to reduce the activity of the transcription factor of interest. Recently, a double-stranded oligonucleotide decoy that includes a first binding site for a first transcription factor and a second binding site for a second transcription factor have been described (see, e.g., US Pub. No. 2018/0298381, Intl. Pub. WO2020/138047 and Intl. Pub. WO2017/043639, each of which is incorporated herein by reference in its entirety). Briefly, a first strand including the sense strand of the first binding site and a second strand including the sense strand of the second binding site are hybridized to form a double strand. Further, the sense strand of the first binding site and the sense strand of the second binding site are at least partly hybridized.

Nuclear factor-κB (NF-κB) is an inducible transcription factor that plays a central role in the inflammatory/immune response. See, for example, Vallabhapurapu S et al. 2009 Annu Rev Immunol 27:693-733. The nuclear translocation of NF-κB enables the transcription factor to bind to its consensus binding site in the promoter region of many genes, including those for inflammatory cytokines and matrix-degrading enzymes (Marcu, K B et al, Curr Drug Targets. 11: 599-613, 2010). The in vitro and in vivo inhibition of cytokines by NF-κB has been shown to be effective in several studies (Akeda et al. Orthop Res Soc. 2006; 413; Fujiwara T et al. ISSLS meeting. 2013; Shoji S et al. ISSLS meeting 2015; and Kato K et al. Orthop Res Soc. 2016). To assess the NF-κB pathway status, several reporter systems have been used to study the activation of NF-κB using NF-κB responsive elements driving the expression of reporter genes, such as Firefly luciferase (Carlsen H et al. 2002 J Immunol 168:1441-6) or eGFP (Magness S T et al. 2004 J Immunol 173:1561-70). This can be achieved by using the lenti virus reporter system for transient transduction or the establishment of pathway sensor cell lines (Landman E B et al. 2014 Cartilage 5:181-9). As disclosed herein, a NF-κB reporter system was applied to stem cells that provides unlimited expansion of the pathway sensor cells. Accordingly, a stem cell, a cell derived therefrom (such as a chondrocyte derived from the stem cell), a cell line of each thereof, a cell population thereof, or a three-dimensional (3D) cell culture thereof comprising the reporter system may be used for assessing the NF-κB pathway status (such as active or not) in vitro, such as for a certain time period, such as at least about 1 day, at least about 2 days, at least about 3 days, at least about 4 days, at least about 5 days, at least about 6 days, at least about 7 days, at least about 10 days, at least about 2 weeks, at least about 3 weeks, at least about 1 month, at least about 2 months, at least about 3 months, at least about 4 months, at least about 6 months, at least about 1 year or longer. In some embodiment the NF-κB reporter system comprises, or consists essentially of, or yet further consists of a vector (for example, a lentivirus) comprising, or consisting essentially of, or yet further consisting of NF-κB responsive elements driving the gene expression of a detectable marker (for example, a Luciferase, an oxidative enzyme that produce bioluminescence). In some embodiments, the stem cell have strong chondrocytic potential, i.e., is capable of deriving and producing chondrocytes under suitable culture condition. In further embodiments, the cell is human embryonic stem-derived progenitor (hEP) cell (4D20.8). 4D20.8 has been shown to have strong chondrocytic potential in a micromass culture system (Sternberg H et al. 2014 Regen Med 9:53-66) and a consistent expansion capacity with chondrogenic potency for over 30 passages. As shown in the Examples as provided herein in addition to Yamada J et al, Orthop Res Soc. Trans. 494, 2018, a human embryonic stem-derived progenitor (hEP)-NF-κB reporter cell line was established. The hEP cell line (4D20.8) has chondrogenic potential under the conditions of a specific culture medium and a micromass culture system (Burns T C et al, Expert Opin Biol Ther. 11:447-61, 2011).

NF-κB decoy is an oligodeoxynucleotide containing NF-κB binding sites which entrap NF-κB subunits (Morishita, R et al. Nat Med. 1997; 3: 894-9). Chimera decoy oligodeoxynucleotide (CDODN, CD-ODN or Chimera decoy), with binding sequences for two different transcription factors (i.e., NF-κB and Ets or E2F), has been developed to improve the efficacy of the decoy oligodeoxynucleotide (ODN) (Mariana K O et al, Adv Polym Sci. 2012) by modulating endogenous transcriptional regulation in multiple pathways. Based on this strategy, a novel chimera decoy ODN that binds to both NF-κB and signal transducer and activator of transcription 6 (STAT6) binding sites has been developed (hereafter referred to as Chimera decoy) (Mariana K O et al, Adv Polym Sci. 2012).

An exemplary double-stranded oligonucleotide decoy described in US Pub. No. 2018/0298381 is a NF-κB/STAT6-15mer-B decoy, which is represented by the following Formula I (SEQ ID NO: 1):

spacer [I]
first strand
5′ GGGATTTCCTgggaa3′
second strand
3′ ccctAAAGGACCCTT5′

Thus, for the NF-κB/STAT6-15mer-B having the structure represented by the Formula [I], the first transcription factor is NF-κB, and the second transcription factor is STAT6. The first and second strands are complementary, and thus the second strand is a complementary strand of the first strand. In the first strand, the sequence GGGATTTCCT (SEQ ID NO: 2), which is designated by capital letters, is the binding site for NF-κB, and in the second strand, the sequence TTCCCAGGAAA (SEQ ID NO: 3), which is designated by capital letters (this sequence is written, in the Formula [I], with its 3′ end on the left since it is a complementary strand; and thus this sequence and the sequence represented in the Formula [I] are the same in fact though they are written in opposite directions), is the binding site for STAT6.

Consensus sequences of transcription factors are often represented by general formulae. The consensus sequence of NF-κB is GGGRHTYYHC (SEQ ID NO: 4) (wherein R, represents A or G, Y represents C or T, and H represents A, C or T), and the consensus sequence of STAT6 is TTCNNNNGAA (SEQ ID NO: 5) (wherein N represents A, G, T or C). Therefore, the binding site GGGATTTCCT (SEQ ID NO: 2) for NF-κB in the first strand is the same as the consensus sequence of NF-κB, except that only one base at the 3′ end mismatches with the consensus sequence of NF-κB. The binding site TTCCCAGGAAA (SEQ ID NO: 3) for STAT6 in the second strand contains the whole of the consensus sequence of STAT6. When describing a base sequence, the base sequence of the sense strand is described, though the binding site for and the consensus sequence of the transcription factor are double-stranded. Thus, the base sequences of the binding sites and the consensus sequences as described above are all the base sequences of the sense strands. Thus, the first strand contains the sense strand of the binding site for NF-κB, and the second strand contains the sense strand of the binding site for STAT6.

An additional exemplary oligonucleotide decoy NF-κB/STAT6-15mer-A) is provided as Formula II (SEQ ID NO: 6):

spacer [II]
first strand
5′ GGGACTTCCCatgaa3′
second strand
3′ ccctGAAGGGTACTT5′

Thus, for the NF-κB/STAT6-15mer-B having the structure represented by the Formula [II], the first transcription factor is NF-κB, and the second transcription factor is STAT6. The first and second strands are complementary, and thus the second strand is a complementary strand of the first strand. In the first strand, the sequence GGGACTTCCC (SEQ ID NO: 7), which is designated by capital letters, is the binding site for NF-κB, and in the second strand, the sequence TTCATGGGAAG (SEQ ID NO: 8), which is designated by capital letters (this sequence is written, in the Formula [II], with its 3′ end on the left since it is a complementary strand; and thus this sequence and the sequence represented in the Formula [II] are the same in fact though they are written in opposite directions), is the binding site for STAT6.

The chimeric decoy of the present disclosure may be a simple double strand, or may be a hairpin or dumbbell (staple) decoy in which one or both ends of each strand are bound via a spacer. Hairpin and dumbbell decoys are preferred because of their higher stability. When comprehensively evaluating the binding activities to transcription factors and the stability, the hairpin decoy is most preferred. Method of making such harpin double stranded chimeric decoys and other nuclease resistance modifications are well-known as described in U.S. Publication No. 2018/0298381.

### Methods and Compositions for Use in the Methods

Previous studies have demonstrated that NF-κB decoy ODN transfection prevents or even restores intervertebral disc degeneration in a rabbit needle puncture disc degeneration model and a rabbit anular (also referred to as annular in the art) puncture model (Akeda K et al, Orthop Res Soc. Trans. 45, 2005; and Akeda et al. Orthop Res Soc. 2006; 413) and also stimulates extra-cellular matrix synthesis (such as the mRNA expression of extracellular matrix genes and proteoglycan synthesis) in an in vitro 4D20.8 micromass culture (Kato K et al, Orthop Res Soc. Trans. 1368, 2016). CDODN has been shown to suppress the NF-κB pathway activated by lipopolysaccharide (LPS) in a 4D20.8 cell micromass culture immediately after Chimera decoy transfection (Yamada J et al, Orthop Res Soc. Trans. 494, 2018). The CDODN was also shown to inhibit cytokine expression and matrix degradation in various tissues. Previous studies have demonstrated that Chimera decoy ODN transfection inhibited inflammatory processes in IL-1 stimulated rabbit synovial tissue and human synovial tissue from osteoarthritis patients in vitro (Miyazaki S et al. Orthop Res Soc. 2017; 538; and Miyazaki S et al. Orthop Res Soc. 2018 387).

In various embodiments, the half-life of an agent (such as a decoy) in a joint is short, such as 1 hours to about 10 hours, see, for example, Jones I A, et al. Nat Rev Rheumatol. 2019 February; 15(2):77-90; Schumacher H R Jr. Arthritis Rheum. 2003 Jun. 15; 49(3):413-20; Gerwin N, et al. Adv Drug Deliv Rev. 2006 May 20; 58(2):226-42; and Owen S G, et al. Br J Clin Pharmacol. 1994 October; 38(4):349-55, each of which is incorporated herein by reference in its entirety. Surprising as disclosed herein, even a short presence of the decoy is still effective in reducing or inhibiting the inflammatory response and in achieving other effects as disclosed herein. Without wishing to be bound by the theory, an agent (such as a decoy) can be diffused faster in a cavity comprising, or consisting essentially of, or alternatively consisting of synovial fluid (also referred to herein as synovia), for example compared to other parts of a subject, therefore resulting in a shorter half-life in such cavity. One of skill in the art would understand that an administration as disclosed herein may be an administration into a cavity comprising, or consisting essentially of, or alternatively consisting of synovial fluid, and the disclosure herein teaches that a single dose to such cavity has a long term effect even though an agent (such as a decoy) has a short half-time in the cavity.

However, it is still unknown how long the effect of a single transfection of decoy ODN lasts. i.e., no long term treatment benefits would be expected, for example, beyond the time point when the effect reaches its peak, or after reducing or inhibiting an initial and acute inflammation. Without wishing to be bound by the theory, hypothesized and tested herein is if the biological effects of a single exposure of CDODN to cells is long enough to suppress the inflammatory response, a single injection, such as intraarticular or intervertebral disc injection, can be effective to modulate a disease status and/or treat a disease. Further hypothesized and shown herein are that a blockage of multiple cytokine-signaling pathways provides a strong inhibitory effect on PG degradation in degenerated IVD cells and that CDODN inhibits inflammatory responses in the IVD leading to the suppression of disc degeneration more effectively than NF-κB decoy. Provided accordingly here are Examples investigating how long the effect of the Chimera decoy lasts using this hEP-NF-κB reporter system, showing results relating to the effects of Chimera decoy ODN on PG degradation in human anulus fibrosus (AF) tissue from patients undergoing discectomy procedures, and comparing the effects of CD-ODN and NF-κB decoy on the morphological and histological changes in a rabbit anular-puncture disc degeneration model.

Accordingly, the disclosure provides a method for the treatment of a disease as disclosed herein, such as spinal pain, low back pain, IVD degeneration, Facet osteoarthritis (OA), or an inflammation leading to each of the disease, a method for regenerating chondrocyte extracellular matrix, synovial joint degeneration or pain, and/or a method for promoting synthesis of proteoglycan. The method includes administering to a subject in need thereof a single dose (e.g., via intra-articular or intervertebral disc injection) comprising, or consisting essentially of, or further consisting of an effective amount of a decoy as disclosed herein, such as a double-stranded oligonucleotide decoy capable of binding to the DNA binding site of NF-κB and to the DNA binding site of signal transducer and activator of transcription 6 (STAT6), thereby treating the disease, such as spinal pain, in the subject.

In various embodiments relating to any aspect or other embodiment(s) as disclosed herein, a single dose refers to a dose given in one single injection. In further embodiments, the injection may be an infusion over a certain time period, such as less than about 1 day, less than about 12 hours, less than about 8 hours, less than about 7 hours, less than about 6 hours, less than about 5 hours, less than about 4 hours, less than about 3 hours, less than about 2 hours, less than about 1 hours, or less. In various embodiments, a single dose refers to one dose given in one single injection to each joint, for example facet joint.

In various embodiments relating to any aspect or other embodiment(s) as disclosed herein, an administration is into a cavity comprising, or consisting essentially of, or alternatively consisting of synovial fluid. In further embodiments, a single dose to such cavity leads to a long term effect even though a decoy has a short half-time in the cavity, for example about 1 hour to about 10 hours.

In various embodiments, the single dose is effective to treat the disease, such as to modulate spinal pain for about 1 day, about 2 days, about 3 days, about 4 days, about 5 days, about 6 days, about 7 days, about 8 days, about 9 days, about 10 days, about 11 days, about 12 days, about 13 days, about 14 days, about 21 days, about 28 days, about 1 week, about 2 weeks, about 3 weeks, about 4 weeks, about 5 weeks, about 6 weeks, about 7 weeks, about 8 weeks, about 9 weeks, about 10 weeks, about 11 weeks, about 12 weeks, about 13 weeks, about 14 weeks, about 15 weeks, about 1 month, about 2 months, about 3 months (or about 12 weeks), about 4 months, about 5 months, about 6 months, about 7 months, about 8 months, about 9 months, about 1 year, or longer. In various embodiments, the single dose is effective to treat the disease, such as to modulate spinal pain for at least about 1 day, at least about 2 days, at least about 3 days, at least about 4 days, at least about 5 days, at least about 6 days, at least about 7 days, at least about 8 days, at least about 9 days, at least about 10 days, at least about 11 days, at least about 12 days, at least about 13 days, at least about 14 days, at least about 21 days, at least about 28 days, at least about 1 week, at least about 2 weeks, at least about 3 weeks, at least about 4 weeks, at least about 5 weeks, at least about 6 weeks, at least about 7 weeks, at least about 8 weeks, at least about 9 weeks, at least about 10 weeks, at least about 11 weeks, at least about 12 weeks, at least about 13 weeks, at least about 14 weeks, at least about 15 weeks, at least about 1 month, at least about 2 months, at least about 3 months (or about 12 weeks), at least about 4 months, at least about 5 months, at least about 6 months, at least about 7 months, at least about 8 months, at least about 9 months, at least about 1 year, or longer. As such, the effect of the double-stranded oligonucleotide decoy remains for over at least 1 week or more, such as 2 weeks or more, 3 weeks or more, 4 weeks or more, 5 weeks or more, 6 weeks or more, 7 weeks or more, 8 weeks or more, 9 weeks or more, 10 weeks or more, 11 weeks or more, 12 weeks or more, 13 weeks or more, 14 weeks or more, 15 weeks or more, 16 weeks or more, 1 month or more, 2 months or more, 3 months or more, 4 months or more, 5 months or more, 6 months or more, 7 months or more, 8 months or more, 9 months or more, or 1 year or more.

In some embodiments, the term effectiveness or a grammatical variation thereof refers to one or more treatment effects of the corresponding disease, such as decreasing the gene expression of pro-inflammatory cytokines, suppressing an inflammatory response, treating intervertebral disc degeneration, regenerating chondrocyte extracellular matrix, promoting synthesis of proteoglycan, treating Facet osteoarthritis (OA), treating low back pain (LBP), and/or treating spinal pain. In some embodiment, treatment effects comprise, or consist essentially of, or further consist of reduced expression, production and/or level of one or more of: a pro-inflammatory cytokine, a pain-related molecules (such as vascular endothelial growth factor (VEGF), prostaglandin-endoperoxide synthase 2 (PTGS2), and nerve growth factor (NGF)), a catabolic molecule (such as ADAM Metallopeptidase With Thrombospondin Type 1 Motif 4 (ADAMTS4)), Matrix Metallopeptidase 3 (MMP3), prostaglandin E2 (PGE2), and/or nitric oxide (NO).

In various embodiments, the decoy has a size of from about 13 mer to about 15 mer. In various embodiments, the decoy has a sequence represented by SEQ ID NO: 1 or 6 or an equivalent thereof. In various embodiments, at least a part of bonds between each nucleotide in the double-stranded oligonucleotide decoy includes a phosphorothioate bond. In various embodiments, at least a part of bonds between each nucleotide in the double-stranded oligonucleotide decoy includes a phosphorothioate bond. In various embodiments, the 5′ end of the decoy is bound, via a linker or directly, to a PLGA nanoparticle. In various embodiments, the decoy is administered directly into a facet joint of the subject. In various embodiments, the decoy is administered via intradiscal injection, or intra-articular injection, or intervertebral disc injection, or epidural injection. In various embodiments, the single dose is effective for about 2 weeks or longer, such as 12 weeks.

The chimeric decoy of the present disclosure can be administered as it is, or can also be administered after it is conjugated with a substance constituting an appropriate drug delivery system (DDS). Thus, in various embodiments, the chimeric decoy is administered without a DDS, thereby reducing the overall cost of therapy.

Examples of DDSs for an oligonucleotide include liposomes containing cationic substances, cell membrane permeable peptides, polymers containing them, and atelocollagen. Besides these, the chimeric decoy can also be conjugated to PLGA (polylactic acid/glycolic acid copolymer) nanoparticles for administration. PLGA nanoparticles are particles having a diameter of tens of nanometers to hundreds of nanometers composed of PLGA. When the chimeric decoy is conjugated to PLGA nanoparticles, the 5′ end of the first strand of the chimeric decoy is preferably conjugated to PLGA nanoparticles via a disulfide linker and an amino linker. This can be carried out, for example, by reacting a PLGA-NHS ester with the chimeric decoy to obtain a PLGA-conjugated chimeric decoy and further making it into a nano-sized particle by utilizing the Marangoni effect.

Following administration of the decoy, to determine the efficacy of the treatment, the present methods encompass determining or measuring the level of low back pain (LBP). In some embodiments, the methods may involve determining or measuring the level of the intervertebral disorder before treatment in order to establish the amount of decoy needed to sufficiently treat the LBP in the subject. In various embodiments, the level of fibrocartilage degrading factors or their precursors, e.g., pro-enzymes, mRNA, etc., can be measured to ascertain the amount of fibrocartilage degradation. Generally, a fibrocartilage-degrading factor encompasses any compound that, when present, leads to the degradation of fibrocartilage tissue in an intervertebral disc. The fibrocartilage-degrading factor can act directly on fibrochondrocytes or fibrocartilage tissue to cause degradation, affect a compound that directly degrades fibrocartilage tissue, or affect a modulator of a compound that degrades fibrocartilage tissue. Fibrocartilage degrading factors include enzymes that directly degrade the cartilage matrix as well as other chemicals that stimulate cartilage degradation, including cytokines such as IL-1. IL-1 appears to indirectly cause fibrocartilage degradation by at least upregulating matrix metalloproteinase activity. Non-limiting examples of methods of measuring fibrocartilage-degrading factors include measuring nitric oxide (NO) production, proteinase detection, or both.

Proteinases, which occupy a specific group of fibrocartilage degrading factors, can be detected in normal and pathological intervertebral discs. These proteinases include, but are not limited to, matrix metalloproteinases (MMPs) and members of the ADAMTS family. Fibrocartilage-degrading factors including proteinases can be detected by any method known in the art. These methods include Western Blot analysis, immunohistochemistry, detection of RNA transcripts, and zymography. The fibrocartilage or fibrochondrocytes from the intervertebral disc can be treated with a fibrochondroprotective agent before measurement of the fibrocartilage degrading factors. Detection can also be conducted before contact, after contact, or both of a fibrocartilage-degrading factor. In various embodiments, the fibrocartilage degrading factors are natural factors.

The route of administration of the chimeric decoy is not particularly limited, but may be preferably parenteral administration such as intravenous administration, intramuscular administration, subcutaneous administration, dermal administration, or direct administration to the target organ or tissue. The dosage is appropriately selected depending on, for example, the target disease, symptoms of the patient, and the route of administration, but usually 0.1 to 10000 nmol, preferably 1 to 1000 nmol, and more preferably 10 to 100 nmol per day for an adult may be administered.

Pharmaceutical compositions of transcription factor inhibitory compounds such as the chimera decoy, can be prepared by mixing one or more transcription factor inhibitory compounds with pharmaceutically acceptable carriers, excipients, binders, diluents or the like, to therapeutically treat, reverse or ameliorate a variety of intervertebral disc disorders and/or LBP and/or another disease as disclosed herein. A therapeutically effective dose refers to that amount of one or more transcription factor inhibitory compounds sufficient to result in amelioration of symptoms of the intervertebral disc disorder and/or another disease as disclosed herein. An effective dose can also refer to the amount of one or more transcription factor inhibitor compounds sufficient to result in prevention of the intervertebral disc disorder and/or LBP and/or another disease as disclosed herein. In some embodiments, the effective dose only partially prevents the intervertebral disc disorder and/or LBP and/or another disease as disclosed herein. In these cases, the disorder of the intervertebral disc and/or another disease as disclosed herein, although they may still exist, are less than the expected intervertebral disc disorder and/or the other disease if no treatment had been given.

The pharmaceutical compositions can be manufactured by methods well known in the art such as conventional granulating, mixing, dissolving, encapsulating, lyophilizing, emulsifying or levigating processes, among others. In certain embodiments, the transcription factor inhibitory compounds, such as the chimera decoy, can be administered in a local rather than a systemic fashion, such as injection as a sustained release formulation. In some embodiments, an effective amount of the transcription factor inhibitory compounds can be administered in any satisfactory physiological buffer such as a phosphate buffer solution (PBS) or in a 5% lactose solution to the pathological intervertebral disc. The dosage forms disclosed in the instant specification are given by way of example and should not be construed as limiting the instant disclosure.

The formulations of the transcription factor inhibitory compounds, such as the chimera decoy, can be designed for to be short acting, fast releasing, long acting, and sustained-releasing as described below. Thus, the pharmaceutical formulations can also be formulated for controlled release or for slow release, such as being contained within a biodegradable matrix or carrier.

The transcription factor inhibitor/decoy in the instant compositions can also exist in micelles or liposomes, or some other encapsulated form, or can be administered in an extended release form to provide a prolonged storage and/or delivery effect. Therefore, the pharmaceutical formulations can be compressed into pellets or cylinders and implanted as stints. Such implants can employ known inert materials such as silicones and biodegradable polymers.

A therapeutically effective dose of a transcription factor inhibitor, such as the chimera decoy, can vary depending upon the route of administration and dosage form. The exact dose is chosen by a physician in view of the condition of a patient to be treated. Doses and administration are adjusted to provide a sufficient level of the active portion, or to maintain a desired effect. Specific dosages can be adjusted depending on conditions of disease, the age, body weight, general health conditions, sex, and diet of the subject, dose intervals, administration routes, excretion rate, and combinations of drugs. A sustained action pharmaceutical composition may be administered repeatedly within a certain interval such as every 3 to 4 days, every week, or once per two weeks (bi-monthly), depending on the half-life and clearance rate of a specific preparation. Guidance for specific doses and delivery methods are provided in publications known in the art. Any of the above dosage forms containing effective amounts are well within the bounds of routine experimentation and therefore, well within the scope of the instant disclosure.

In a further aspect, provided is a composition for use in a method as disclosed herein comprising, or consisting essentially of, or further consisting of one or more of: a chimera decoy as disclosed herein, a polynucleotide encoding the chimera decoy, and/or a vector comprising the polynucleotide; and a carrier, such as pharmaceutical acceptable carrier. In yet a further aspect, provided is a kit comprising, or consisting essentially of, or further consisting of one or more chimera decoys as disclosed herein and an instruction for use in a method as disclosed herein. In further embodiments, the kits can also include one or more reagents, buffers, media, proteins, analytes, labels, cells, computer programs for analyzing results, and/or disposable lab equipment, such as culture dishes or multi-well plates, in order to readily facilitate implementation of the present methods. Solid supports can include beads, culture dishes, multi-well plates and the like.

### EXAMPLES

The following examples are intended to illustrate but not limit the disclosure.

### Example 1

### Evaluation of the Long-Term Effect of a Single Transfection Treatment with Chimera Decoy of Chondrogenic Cells Cultured in a High-throughput Three-Dimensional System

### Methods

hEP-NF-κB reporter cell line: The reporter cell line was derived from the 4D20.8 cell line by transfection with firefly luciferase expressing the NF-κB reporter lentiviral vector (Cignal Lenti C Reporter (Luc), Qiagen) and constitutively the Renilla luciferase expressing lentiviral vector (Cignal Lenti Renilla Control (luc), Qiagen).

Micromass culture: 4D20.8 cells (P7) were expanded in DMEM with 20% fetal bovine serum (FBS) and 2 mM L-glutamine at 10% O₂. After trypsinization of cells (P11), a cell suspension (0.2×10⁶ cells in 20 μl) in growth media was placed as a micromass drop on a non-tissue culture-coated 96-well plate (V-shaped) and cultured at 5% O₂. After 24 hours, 100 μl of chondrogenic media (BioTime, Alameda Calif.) with 10 ng/ml of bone morphogenetic protein 4 (BMP 4) and 10 ng/ml of transforming growth factor β (TGF-β) was added to each well to induce chondrogenic differentiation (Kato K et al, Orthop Res Soc. Trans. 1368, 2016).

Chimera decoy ODN transfection of cell pellet: After micromass culture for 14 days, cell pellets were pre-treated with phosphate-buffered saline (PBS) or CDODN (10 μM) (AnGes, Inc, Osaka Japan) for 24 hours.

LPS stimulation: After pre-treatment with PBS or CDODN, cells were further divided into two subgroups (immediately after transfection group and 1 week after transfection group). Cells were cultured in the same media with or without LPS (1 μg/ml) for 12 hours on day 1. After LPS stimulation, cell pellets were disrupted by cell lysis buffer and the supernatant was used for the luciferase assay. After one week of post-transfection culture in the absence of ODN, the same experiment was repeated to assess the effect of CDODN.

Firefly luciferase assay: The luciferase assay was performed using D-luciferin for Firefly (ThermoFisher Scientific). Luminescence was measured with the Synergy HTX (BioTek).

### Results:

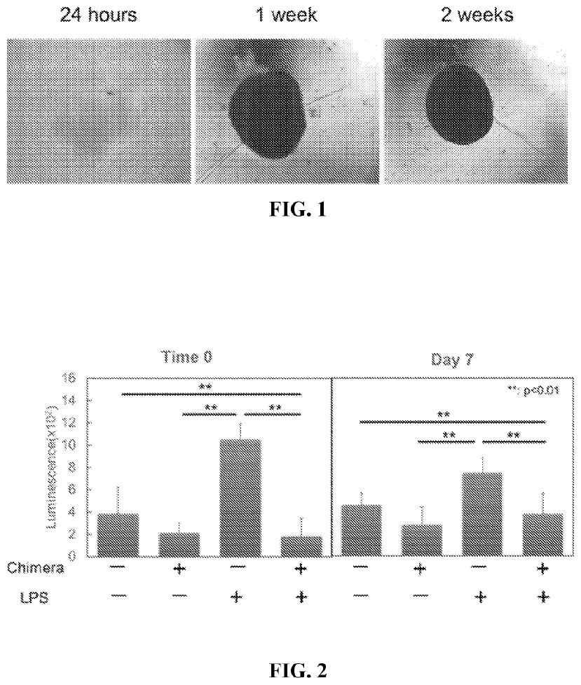

Micromass culture: Twenty-four hours after inoculation, the cells started to show signs of aggregation. After one week of culture, the cells showed a typical spherical pellet structure in all wells. At the two week time point, the pellet size was slightly reduced (FIG. 1).

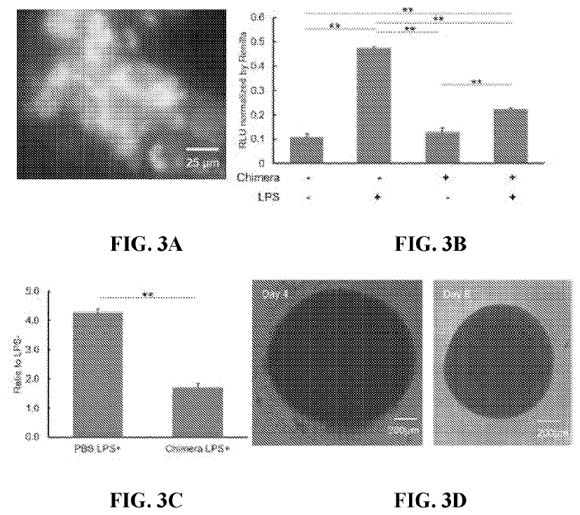

Luciferase activity after LPS stimulation (FIG. 2): Time 0: LPS significantly stimulated luciferase activity in the control with LPS group (274% of the control culture, p<0.01). This was significantly reduced when the cells were pre-treated with CDODN (suppressed to 47% of the control group, P<0.01). The constitutive activity of luciferase did not change with CDODN. There were no significant differences among the three other groups.

Day 7: Similar to the results at Time 0, luciferase activity of the control with LPS group was significantly higher than that of all other groups. LPS stimulated the control culture up to 163%. This effect was completely abolished by a single treatment with CDODN for 24 h (suppressed to 82% of the Control group, P<0.01).

### Discussion:

Presence of an ODN in a subject for a long period was considered as required to achieve a long term effect. But, surprisingly, the result here indicates one exposure is effective, for at least over one week. This is the first time the long-term effect of a single ODN transfection was evaluated in chondrocytic cells. The results reveal that the effect of CDODN remained for over one week. This approach was achieved by using the hEP-based pellet culture system with the lentivirus transfection of a reporter gene. This cell line can be easily cryo-preserved, used for assays, and because the pellet culture can be maintained for over 4 weeks, further long term analyses can be performed.

After a single injection of decoy ODN, the first phase of its half—life (distribution) was shown to be 11.9 hours and the second phase of its half-life (elimination) was shown to be 618 hours after a single injection of decoy ODN (Kato K et al, Orthop Res Soc. Trans. 1368, 2016). The half-life of CDODN is predicted to be similar to that of decoy ODN, based on its molecular structure. Importantly, this study further revealed that a single exposure to CDODN was effective for over 7 days. These results support the embodiment of the disclosure where a single injection of decoy ODN or CDODN has clinical applications.

The effect of chimera decoy lasted for at least 1 week using the hEP-NF-κB system. This result indicates the usefulness of chimera decoy ODN in clinical practice.

### Example 2

### Effect of Chimera Decoy

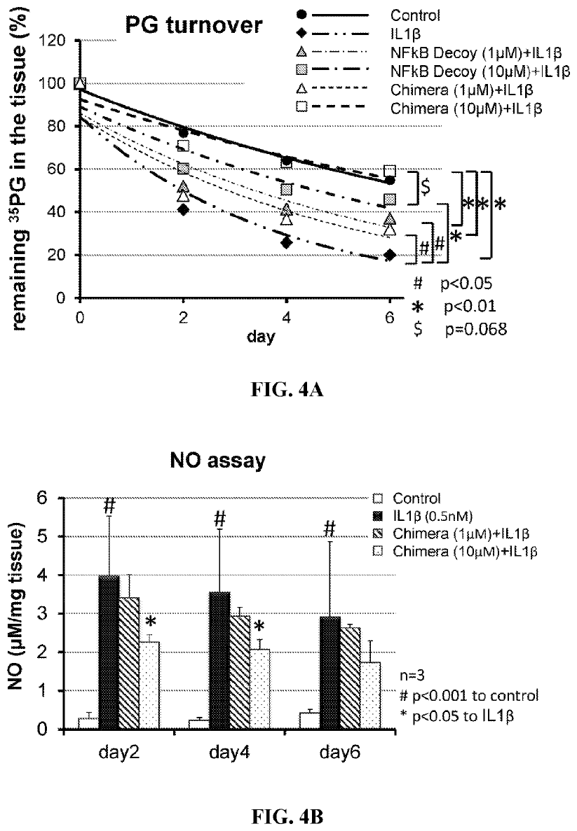

This study established, for the first time, a hEP-NF-κB reporter cell line using 4D20.8 human embryonic progenitor cell that have chondrogenic potential. Fluoroscopic images revealed FAM chimera decoy (C-Decoy) was seen in the cytoplasm of most cells for 3 hours of FAM chimera ODN incubation (FIG. 3A) (Yamada J, Trans Orthop Res Soc:0494, 2018). C-Decoy was successfully transfected and the NF-κB pathway was clearly activated by LPS stimulation (FIGS. 3B-3C), confirming the validity of the reporter system. C-Decoy significantly inhibited NF-κB activation; this suggests that this system can be used for validation of the effects of a NF-κB pathway modifier (FIG. 3B). This hEP-NF-κB reporter cell line can be consistently expanded and used for 3D pellet culture, which maintains chondrocytic properties. The results provide a high throughput 3D NF-κB reporter system where various biologics/compounds can be tested under relevant biological conditions. In this study, this established reporter cell line is utilized to identify the longevity of the inhibitory effect of C-Decoy. This result reveals the biological activity of C-Decoy incorporated into cells; therefore, the long-term effect of a single injection of C-Decoy to facet joints can be justified.

For the monolayer group, hEP-NF-κB reporter cells (2.0×10⁴ cells/well) is seeded in 96-well plates as monolayer cultures. At 80% confluency, cells are used as the monolayer culture group. For the pellet culture, after trypsinization of expanded hEP-NF-κB reporter cells, a cell suspension (0.5×10⁶ cells in 20 μl) is inoculated into the bottom of each well of a non-tissue culture-coated 96-well plate (V-shaped) and cultured at 5% CO₂/5% O₂ without centrifugation to form pellets. After two hours, 100 μl chondrogenic media with 10 ng/ml bone morphogenetic protein4 (BMP4) and 10 ng/ml transforming growth factor β (TGF-β) are added to each well to induce chondrogenic differentiation and culture for 14 days.

Cells or pellets are cultured in the presence of PBS or C-Decoy (10 μM) for 24 hours for transfection. After pretreatment, cells are cultured in the complete media for 6h, 1, 3, 7, 14, 21 and 28 days. At the end of culture period cells are incubated in the serum-free media (with ITS) with or without lipopolysaccharide (LPS) (1 μg/ml). After LPS stimulation, cells are disrupted by cell lysis buffer and subjected to a freeze and thaw cycle. The luciferase assay is performed using D-luciferin for Firefly luciferase (ThermoFisher Scientific). For selected experiments, a co-transfected cell line is used with Renilla luciferase. Coelenterazine is used for Renilla luciferase (ThermoFisher Scientific). Luminescence is measured with the Synergy HTX (BioTek). Renilla is used as a normalization control.

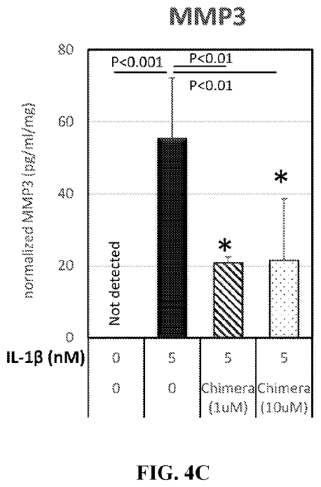

A study was conducted to investigate the roles of STATE, as well as NF-κB, in inflammatory responses of chondrocytes using rabbit articular cartilage (FIG. 4) and to investigate the effect of Chimera decoy on proteoglycan (PG) turnover, and matrix metalloproteinase 3 (MMP3) and nitric oxide (NO) production in IL-1-stimulated rabbit articular cartilage, and to compare these to the effects of conventional NF-κB decoy ODN, which includes only the NF-κB binding site (NF-κB decoy).

Treatment with 10 μM Chimera decoy effectively attenuated the inflammatory response stimulated by IL-1β. Surprisingly, PG turnover accelerated by IL-1β was almost completely restored to the level of the control by treatment with 10 μM Chimera decoy (FIG. 4), while treatment with 1 μM Chimera decoy had little effect. Inflammatory markers, such as NO and MMP3, stimulated by IL-1β, were also reduced by Chimera decoy (FIGS. 4B-4C).

It was determined that the manipulation of the signaling pathway using NF-κB decoy which is involved in signaling of IL-1 and TNF-α, could delay or attenuate facet joint articular cartilage degeneration.

After a two-day pre-culture period in complete medium, the cartilage explants were cultured in DMEM/F12 with 10% FBS in the absence or presence of NF-κB decoy (10 μM) or scrambled decoy (10 μM; as the negative control; AnGes MG, Inc., Japan.) for seven days with daily changes of media. At the end of the seven-day culture period, PG synthesis, content and turnover were assessed.

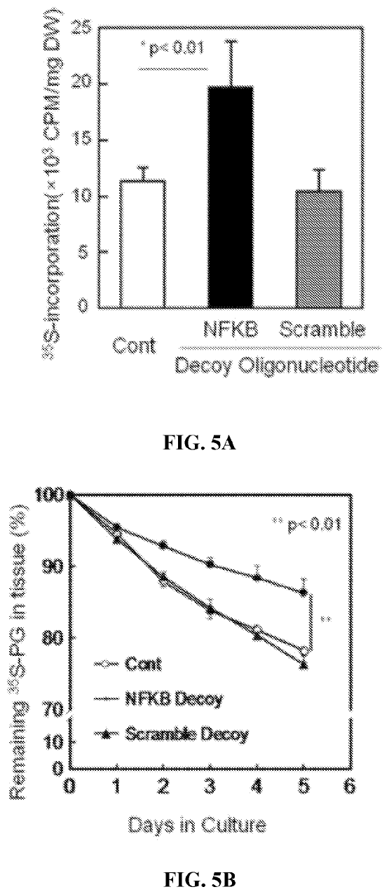

Compared to the control, treatment of the cartilage explants with NF-κB decoy for seven days significantly stimulated PG synthesis expressed per dry weight (+74% p<0.01, n=8 donors). The scrambled decoy did not show an effect on PG synthesis (FIG. 5A). Treatment with NF-κB decoy for seven days significantly slowed the rate of PG disappearance from the cartilage explants (+11% on day 5 of treatment with NF-κB decoy, n=4 donors, p<0.01). The scrambled decoy did not have an effect of PG turnover (FIG. 5B). The release of MMP-3 was significantly inhibited by treatment with NF-κB decoy (−65%, p<0.05) and NO levels in the media also showed a similar tendency (−73%, p=0.067). These results indicate that NF-κB decoy can stimulate PG synthesis and inhibit the degradation of PGs in an explant culture system of human facet joint cartilage. Scrambled decoy did not have an effect on PG turnover (FIG. 5B). The chimera decoy as disclosed herein is tested using the same experimental setting, optionally having the NF-κB decoy serving as a comparison. The data is obtained and plotted.

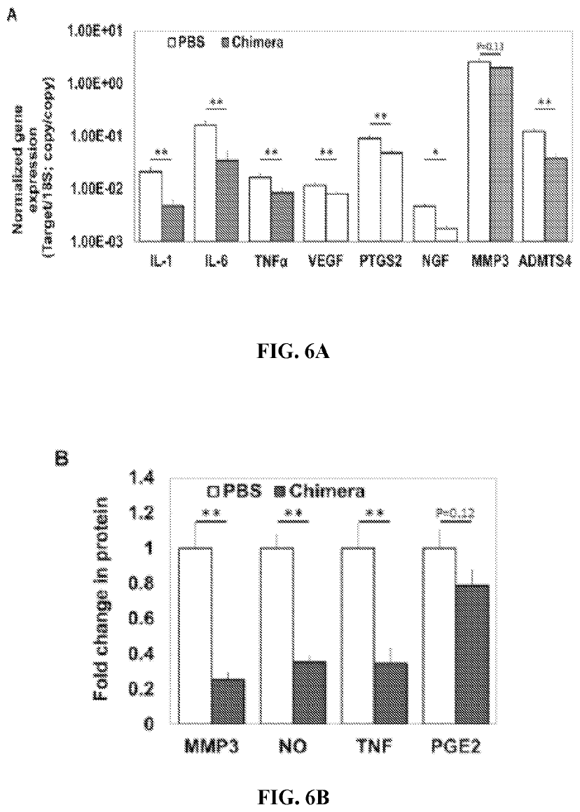

A further study was performed to assess the anti-inflammatory effects of Chimera Decoy on the gene expression and protein release of pro-inflammatory cytokines and pain markers in rabbit (Miyazaki S et al. Trans Orthop Res Soc:0538, 2017) and human osteoarthritic synovium (Miyazaki S et al. Trans Orthop Res Soc:0387, 2018) and to elucidate the possibility for future clinical use. This study demonstrated that treatment with Chimera Decoy carrying two binding sites (NF-κB, STAT6) resulted in significant inhibition of gene expressions of the inflammatory cytokines IL-1β, IL-6, and TNFα and the pain-related molecules VEGF, PTGS2, and NGF, as well as the catabolic molecule, ADAMTS4 (FIG. 6A) (Miyazaki S et al. Trans Orthop Res Soc:0387, 2018). The synovial production of MMP3, NO and TNFα was significantly suppressed by treatment with Chimera Decoy (FIG. 6B). The data suggested that Chimera Decoy was effective at inhibiting the pro-inflammatory factors and pain-related molecules that promote angiogenesis, innervation, and catabolic factors in humans.

However, it is not known how long the effect of a single exposure of C-Decoy continues. Therefore, a long-term effect study is performed using human facet joint tissues and osteoarthritic synovial tissue from patients.

Long-term effects of C-Decoy are tested on NO and cytokines and PGE2 released from synovial tissue, and on the mRNA expression of TNF-α, IL-1, IL-6, catabolic enzymes and matrix components. In addition, the effect of C-Decoy on facet cartilage metabolism in human cadaveric tissues is assessed to shed light on the post-injection cartilage metabolism. Two approaches are taken to reveal these questions using human synovium tissue or cells from patients (readily available) or human facet cartilage, joint tissues from donors.

Knee OA synovia is obtained from patients undergoing total knee arthroplasty. Approximately 100-200 mg of synovial explants per well is pre-cultured in DMEM/F12/10% fetal bovine serum (FBS) media. After the pre-culture, synovia is cultured in the presence/absence of C-Decoy in the culture media for 24 hours and then cultured in the complete media for various lengths. After 1, 2 and 4-week, synovia is serum-starved in DMEM/F12/1% insulin-transferrin-selenite (ITS) medium for 2 hours, and then cultured in the same media in the presence of PBS (vehicle) or IL-1 for 24 hours. Some experiments are performed using monolayer culture of cryopreserved OA synovial cells using the same protocol.

After 24 hours of treatment, tissues are disrupted using stainless steel beads and total RNA extracted using the RNeasy Lipid Tissue Mini Kit (Qiagen). For monolayer, an RNeasy mini kit is used to extract total RNA with a tissue shredder. Whole transcriptome cDNA libraries are synthesized using the QuantiTect Whole Transcriptome kit (Qiagen). Alternatively, cDNA is synthesized using QuantiTect Reverse Transcription Kit (Qiagen). Gene expressions of IL-10, IL-6, TNFα, vascular endothelial growth factor (VEGF), prostaglandin-endoperoxide synthase 2 (PTGS2), nerve growth factor (NGF), matrix metalloprotainase-3 (MMP3), and a disintegrin and metalloproteinase with thrombospondin-like motif-4 (ADAMTS4) are analyzed using quantitative real-time polymerase chain reaction (qPCR) with standards. 18S is used as an endogenous control.

The levels of MMP3, nitric oxide (NO), TNFα, and prostaglandin E2 (PGE2) in conditioned media after 24 hours are measured by ELISA (R&D) and normalized by tissue wet weight.

All data is expressed as mean and standard error (SE). Normalized gene expressions are analyzed after log conversion. The data is analyzed using two-way ANOVA (patient: random factor; treatment: fixed factor). P values less than 0.05 are regarded as statistically different.

A total of 6-10 series of human cadaveric spine tissues (cartilage and synovium), are used to test the effects of C-Decoy on the mRNA expression of TNF-α, IL-1, IL-6 and catabolic enzymes (other parameters may be assessed by request by Anges MG). The effect of C-Decoy on proteoglycan synthesis and turnover is also assessed. The long-term effect of C-Decoy is assessed.

Human spines (6-10 donors) are provided from the sponsor through a regional organ bank; the grade of degeneration of discs and facet joints is assessed by plain X-ray. After removal of the paravertebral muscle and posterior component by cutting the pedicle using a lambotte osteotome, the facet joints are exposed by a sharp dissection of the facet joint capsule. After gross examination of the joint surface, normal or mildly degenerative cartilage is sharply dissected by surgical scalpels. Particular attention is paid to avoiding contamination by synovial tissues. Tissues from 8 or 10 facet joints from a single donor are pooled, cut into 5 mm×5 mm pieces and cultured in medium (DMEM/F12 supplemented with 10% fetal bovine serum (FBS) and 25 μg/ml ascorbic acid) overnight. The synovial capsule is also cultured. The tissue is treated with C-Decoy or scrambled decoy for 24 hours and further cultured for 1-28 days. At the end of culture, the media is changed to serum-free media with ITS and culture for 24-48 hours. In some embodiments, tissues are stimulated with IL-1 (10 ng/ml) based on the preliminary results. Media is collected for MMP3, NO and other protein analyses.

Tissue is pulverized or homogenized, and mRNA extracted using Qiazol/Chloroform followed by MiniElute column purification. cDNA is generated using either the QuantiTect Reverse Transcription Kit or the QuantiTect Whole Transcriptome Kit. Q-PCR is performed using gene-specific primers for cytokines (IL-1β, TNF-α, IL-6), matrix-degrading enzymes (a disintegrin and metalloproteinase with thrombospondin motifs-4 (TS4), and MMP3)) and pain molecule (nerve growth factor (NGF)). 18s rRNA is used as the internal control.

Levels of MMP-3, PGE2, and TNF-α in the media are measured to confirm the results at the protein level using commercially available kits. The level of NO is measured using a commercially available kit.

Cartilages are incubated for 16 h in complete medium containing ³⁵S-sulfate (20 μCi/ml). After washing to remove the unincorporated radioisotope, the tissue is cultured for 6 days in isotope-free medium with/without IL-1β (5 ng/ml). The medium in all cases is changed and collected for the measurement of ³⁵S-PGs. At the end of culture, tissues are collected and the content of ³⁵S-PGs in the tissue and the spent medium fraction are measured using a rapid filtration assay (Masuda K et al. Anal Biochem 217:167-75, 1994). For each set of conditions (i.e., with or without decoys), the amount of radiolabeled PGs remaining in tissues is plotted against time of chase to measure the average half-life of ³⁵S-PGs in tissue. The data is fitted to the single exponential decay equation: y=ae−bx+ce−dx+e and the half-life is calculated based on b and d (Mok S S et al. Biol Chem 269:33021-7, 1994).

The synthesis of PGs is assessed in explants of cartilage tissues as previously described by determining the content of radiolabeled ³⁵S-PG.

### Example 3

### Newly Engineered Chimera Decoy Oligodeoxynucleotide and Proteoglycan Degradation in the Human Intervertebral Disc

### Methods

Annulus fibrosus (AF) tissue for organ culture is obtained from patients undergoing lumbar spine surgery. The tissue is washed, cut into 3 mm pieces, and incubated as explant cultures in DMEM with 20% fetal bovine serum (FBS) and 2 mM L-glutamine at 5% O₂. The number of samples ranged from 3 to 5 in each group depending on the size of the harvested IVD disc tissue.

PG turnover: To evaluate PG degradation, the AF tissue is pre-labeled with 20 μCi/ml ³⁵S-sulfate for 16 hours. After labeling, samples are washed 5 times with DMEM/F12 containing 1.5 mM SO₄ for 10 minutes each. The explants are cultured in DMEM/F-12 media with 20% fetal bovine serum (FBS) in the presence or absence of NF-κB decoy ODN (10 μM) (AnGes Inc., Tokyo, Japan) or Chimera decoy ODN (10 μM) (AnGes Inc.) for about 24 hours and culture in medium, free of the treatment for about 5 days. In a comparison set, the explants are cultured in DMEM/F-12 media with 20% fetal bovine serum (FBS) in the presence or absence of NF-κB decoy ODN (10 μM) (AnGes Inc., Tokyo, Japan) or Chimera decoy ODN (10 μM) (AnGes Inc.) for up to 6 days. The media was replaced every other day with the same treatment media and the cultured media was collected. The culture media is collected. The tissue is collected at the end of incubation period and digested with papain. The amount of ³⁵S-PGs in the media and digests is measured by a rapid Alcian blue filtration assay (Masuda K et al. J Orthop Res. 2003; 21: 922-30). The ³⁵S-PGs remaining over total ³⁵S-PGs synthesized is assessed; The ratio to control for each patient is calculated by dividing the average of the treatment group by the average of the control group. The loss of PG and PG synthesis are assessed.

NF-κB activation and LPS stimulation are performed and evaluated as described in Examples 1 and 2.

Statistical analyses: One-way and two-way ANOVA with Fisher PSLD test as a post hoc test. The results are expressed as mean±standard error.

### Example 4

### Chimera Decoy Oligodeoxynucleotide and Intervertebral Disc Degeneration in a Rabbit Anular-Puncture Model

### Methods

Rabbit Anular-puncture Disc Degeneration Model (Masuda K et al. Spine. 2005; 30: 5-14): Surgeries and Injection of CD-ODN: Under general anesthesia, lumbar IVDs of female New Zealand white rabbits (n=64, five months-old) are exposed, and an anular puncture (18-gauge, 5 mm depth) is performed at two non-continuous discs (L2/3 and L4/5), with the disc (L3/4) between the punctured discs left intact as a control. Four weeks after the initial puncture, either the vehicle (phosphate-buffered saline (PBS); 10 μL per disc), NF-κB Decoy ODN (100 μg in 10 μL PBS per disc) or CD-ODN (10 or 100 μg in 10 μL PBS per disc) is injected into the center of the nucleus pulposus (NP) using a fine tip 26-gauge needle (XX*MS16, Ito Corporation, Shizuoka, Japan) attached to a MS*GFN25 syringe (Ito Corporation). Rabbits are euthanized at 16 weeks after the initial anular puncture (12 weeks after the injection). Other sets of rabbits treated as disclosed above are euthanized at more than 16 weeks after the initial anular puncture (more than 12 weeks after the injection), such as about 5 months, about 6 months, about 7 months, about 8 months, about 9 months, or about 1 year or longer after the initial anular puncture (i.e., about 4 months, about 5 months, about 6 months, about 7 months, about 8 months, or about 11 months or longer after the injection).

Radiographic analysis of disc height index (DHI): Lateral radiographs of the lumbar spine are obtained at two-week intervals up to 16 weeks or later for example as provided above after the initial puncture. IVD height is expressed as DHI, as previously described (Mwale F et al. Arthritis Res Ther. 2011; 13: 120). The average percent change in DHI of injected discs (both L2/3 and L4/5) is calculated for each postoperative disc as a ratio to its preoperative DHI (% DHI=(postoperative DHI/preoperative DHI)×100) and further normalized to the DHI of the non-punctured disc (L3/4): (Normalized % DHI=(punctured % DHI/non-punctured % DHI)×100) (Mwale F et al. Arthritis Res Ther. 2011; 13: 120). All radiographs are assessed by an observer blinded to this experiment.

Magnetic resonance imaging (MRI) analyses: After sacrifice at 16 weeks or later for example as provided above, MRI examinations on isolated spine segments are performed using a 7-Tesla BioSpec 70/30 (BRUKER, Billerica, Mass., USA). The average degeneration grade of injected discs (L2/3 and L4/5) is calculated according to Pfirrmann grade (Pfirrmann C W et al. Spine. 2001; 26: 1873-8) using T2 weighted sagittal images; the evaluations are performed by two observers blinded to experimental groups.

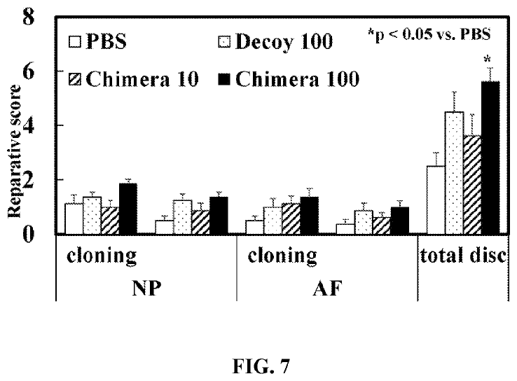

Histological analyses: Midsagittal sections (5 μm) of each experimental IVD from sacrifices at 16 weeks or later for example as provided above are stained with either Hematoxylin and Eosin or Safranin-O. An observer blinded to this experiment analyzes the histologic sections and grades them using an established protocol (Chujo T et al., Spine. 2006; 31:2909-17). In addition, cellular changes including cell cloning or the presence of chondrocyte-like cells in either the inner or outer anulus fibrosus (AF) or in the NP are analyzed as a reparative scale (Chujo T et al. Spine. 2006; 31:2909-17). An exemplified result for data obtained from at 16 weeks are plotted in FIG. 7.

### Equivalents

Unless otherwise defined, all technical and scientific terms used herein have the same meaning as commonly understood by one of ordinary skill in the art to which this technology belongs.

The present technology illustratively described herein may suitably be practiced in the absence of any element or elements, limitation or limitations, not specifically disclosed herein. Thus, for example, the terms “comprising,” “including,” “containing,” etc. shall be read expansively and without limitation. Additionally, the terms and expressions employed herein have been used as terms of description and not of limitation, and there is no intention in the use of such terms and expressions of excluding any equivalents of the features shown and described or portions thereof, but it is recognized that various modifications are possible within the scope of the present technology claimed.

Thus, it should be understood that the materials, methods, and examples provided here are representative of preferred aspects, are exemplary, and are not intended as limitations on the scope of the present technology.

The present technology has been described broadly and generically herein. Each of the narrower species and sub-generic groupings falling within the generic disclosure also form part of the present technology. This includes the generic description of the present technology with a proviso or negative limitation removing any subject matter from the genus, regardless of whether or not the excised material is specifically recited herein.

In addition, where features or aspects of the present technology are described in terms of Markush groups, those skilled in the art will recognize that the present technology is also thereby described in terms of any individual member or subgroup of members of the Markush group.

All publications, patent applications, patents, and other references mentioned herein are expressly incorporated by reference in their entirety, to the same extent as if each were incorporated by reference individually. In case of conflict, the present specification, including definitions, will control.

Other aspects are set forth within the following claims.

Although the disclosure has been described with reference to the above examples, it will be understood that modifications and variations are encompassed within the spirit and scope of the disclosure. Accordingly, the disclosure is limited only by the following claims.

1. A method for the treatment of intervertebral disc degeneration comprising administering to a subject in need thereof a single dose comprising an effective amount of a double-stranded oligonucleotide decoy capable of binding to the DNA binding site of Nuclear factor-κB (NF-κB) and to the DNA binding site of signal transducer and activator of transcription 6 (STAT6), wherein the single dose is effective for over 1 week, thereby treating intervertebral disc degeneration in the subject.

2. (canceled)

3. The method of claim 1, wherein the decoy has a sequence represented by SEQ ID NO: 1 or 6 or an equivalent thereof.

4. (canceled)

5. The method of claim 1, wherein the 5′ end of the decoy is bound, via a linker or directly, to a PLGA nanoparticle.

6-7. (canceled)

8. The method of claim 1, wherein the over 1 week comprises treating the disease for at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, or at least about 13 weeks, or at least about 14 weeks, or at least about 15 weeks, or at least about 4 months, or at least about 5 months, or at least about 6 months, or at least about 7 months, or at least about 8 months, or at least about 9 months, or at least about 1 year or longer.

9. A method for regenerating a chondrocyte extracellular matrix, comprising administering to a subject in need thereof a single dose comprising a double-stranded oligonucleotide decoy capable of binding to the DNA binding site of NF-κB and to the DNA binding site of signal transducer and activator of transcription 6 (STAT6), wherein the single dose is effective for over 1 week, thereby regenerating a chondrocyte extracellular matrix in the subject.

10-11. (canceled)

12. The method of claim 9, wherein the decoy has a sequence represented by SEQ ID NO: 1 or 6.

13-15. (canceled)

16. The method of claim 9, wherein the over 1 week comprises for at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, or at least about 13 weeks, or at least about 14 weeks, or at least about 15 weeks, or at least about 4 months, or at least about 5 months, or at least about 6 months, or at least about 7 months, or at least about 8 months, or at least about 9 months, or at least about 1 year or longer.

17. A method for promoting the synthesis of proteoglycan in intervertebral disc cells of a subject, comprising administering to a subject in need thereof a single dose comprising a double-stranded oligonucleotide decoy capable of binding to the DNA binding site of NF-κB and to the DNA binding site of signal transducer and activator of transcription 6 (STAT6), wherein the single dose is effective for over 1 week, thereby promoting the synthesis of proteoglycan in intervertebral disc cells of the subject.

18-19. (canceled)

20. The method of claim 17, wherein the decoy has a sequence represented by SEQ ID NO: 1 or 6 or an equivalent thereof.

21-23. (canceled)

24. The method of claim 17, wherein the over 1 week comprises for at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, or at least about 13 weeks, or at least about 14 weeks, or at least about 15 weeks, or at least about 4 months, or at least about 5 months, or at least about 6 months, or at least about 7 months, or at least about 8 months, or at least about 9 months, or at least about 1 year or longer.

25. A method for the treatment of spinal pain or low back pain comprising administering to a subject in need thereof a single dose comprising a double-stranded oligonucleotide decoy capable of binding to the DNA binding site of NF-κB and to the DNA binding site of signal transducer and activator of transcription 6 (STAT6), wherein the single dose is effective for over 1 week, thereby treating spinal pain in the subject.

26. (canceled)

27. The method of claim 25, wherein the decoy has a sequence represented by SEQ ID NO: 1 or 6 or an equivalent thereof.

28. (canceled)

29. The method of claim 25, wherein the 5′ end of the decoy is bound, via a linker or directly, to a PLGA nanoparticle.

30-31. (canceled)

32. The method of claim 25, wherein the over 1 week comprises for at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, or at least about 13 weeks, or at least about 14 weeks, or at least about 15 weeks, or at least about 4 months, or at least about 5 months, or at least about 6 months, or at least about 7 months, or at least about 8 months, or at least about 9 months, or at least about 1 year or longer.

33. A method for reducing or suppressing an inflammatory response comprising administering to a subject in need thereof a single dose comprising a double-stranded oligonucleotide decoy capable of binding to the DNA binding site of NF-κB and to the DNA binding site of signal transducer and activator of transcription 6 (STAT6), wherein the single dose is effective for over 1 week, thereby reducing or suppressing the inflammatory response in the subject.

34-35. (canceled)

36. The method of claim 33, wherein the decoy has a sequence represented by SEQ ID NO: 1 or 6 or an equivalent thereof.

37-40. (canceled)

41. The method of claim 33, wherein the over 1 week comprises for at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, or at least about 13 weeks, or at least about 14 weeks, or at least about 15 weeks, or at least about 4 months, or at least about 5 months, or at least about 6 months, or at least about 7 months, or at least about 8 months, or at least about 9 months, or at least about 1 year or longer.

42. A method for the treatment of Facet osteoarthritis (OA), comprising administering to a subject in need thereof a single dose comprising a double-stranded oligonucleotide decoy capable of binding to the DNA binding site of NF-κB and to the DNA binding site of signal transducer and activator of transcription 6 (STAT6), wherein the single dose is effective for over 1 week, thereby treating the OA in the subject.

43. (canceled)

44. The method of claim 42, wherein the decoy has a sequence represented by SEQ ID NO: 1 or 6 or an equivalent thereof.

45-47. (canceled)

48. The method of claim 42, wherein the over 1 week comprises for at least about 2 weeks, or at least about 3 weeks, or at least about 4 weeks, or at least about 5 weeks, or at least about 6 weeks, or at least about 7 weeks, or at least about 8 weeks, or at least about 9 weeks, or at least about 10 weeks, or at least about 11 weeks, or at least about 12 weeks, or at least about 13 weeks, or at least about 14 weeks, or at least about 15 weeks, or at least about 4 months, or at least about 5 months, or at least about 6 months, or at least about 7 months, or at least about 8 months, or at least about 9 months, or at least about 1 year or longer.

49. (canceled)
